(19) United States
(12) Patent Application Publication
(10) Pub. No.: US 2003/0170245 A1
(43) Pub. Date: Sep. 11, 2003

(54) Activation of matriptase and diagnostic and therapeutic methods based thereon

(76) Inventors: Robert B. Dickson, Kensington, MD (US); Chen-Yong Lin, Falls Church, VA (US); Christelle Benaud, Grenoble (FR); Michael Oberst, Chevy Chase, MD (US)

Correspondence Address: PILLSBURY WINTHROP, LLP, P.O. BOX 10500, MCLEAN, VA, 22102, US

(21) Appl. No.: 10/087,987

(22) Filed: Mar. 5, 2002

(51) Int. Cl. A61K 39/395; G01N 33/574

(52) U.S. Cl. 424/155.1; 435/7.23

(57) ABSTRACT

The present invention provides an in vitro method of diagnosing the presence of a pre-malignant lesion, a malignancy, or other pathologic condition, in a subject, which is characterized by the presence of activated matriptase including the steps of: (A) obtaining a biological sample from a subject that is to be tested for a pre-malignant lesion, a malignancy, or other pathologic condition; B) exposing the biological sample to a detectable agent which recognizes and binds to activated matriptase; and (D) determining whether said detectable agent is bound to the biological sample. Preferably, the detectable agent is an antibody which specifically binds to activated matriptase. More preferably the antibody is selected from M69 and M123. The invention also provides a method of treating malignancies, pre-malignant conditions, and pathologic conditions in a subject which are characterized by the activated form of matriptase including administering a therapeutically effective amount of an agent capable of blocking the activity of active matriptase.

### BACKGROUND OF THE INVENTION

1. Field of the Invention

This invention relates to the field of diagnosis and treatment of cancer, particularly breast cancer or other conditions through the detection, inhibition or induction of the activity of proteolytic enzymes.

2. Summary of the Related Art

It has long been proposed that metastasis is a multi-step process. This includes the breakdown of the basement membrane, detachment of cancer cells from the primary tumor, invasion into the stroma, intravasation into blood vessels, survival in the blood stream, extravasation through target organ blood vessels, and the establishment and proliferation of cancer cells in remote tissues. To accomplish these events, cancer cells must acquire an enhanced ability to migrate through and degrade extracellular matrix components. An array of extracellular matrix-degrading proteases and cell motility factors have been characterized and implicated in cancer invasion and metastasis¹. Among the protease systems, the plasmin/urokinase type plasminogen activator (uPA) system²⁻⁶, and the matrix metalloproteases (MMPs)⁷⁻¹² have received the most attention. Although these ECM-degrading proteases have been implicated in breast cancer invasion and metastasis, they are mainly expressed by stromal components of human breast tumors^{9;11; 13-16}. The stromal origins of these extracellular matrix-degrading proteases in breast cancer suggests that malignant invasion is an event which depends at least in part upon a stromal-epithelial interaction¹⁷. Furthermore, growth and motility factors secreted by stromal cells may also contribute to the ability of cancer cells to migrate through the extracellular matrix. Hepatocyte growth factor (HGF)/scattering factor (SF) is one of these mesenchymal cell-derived proteins. Upon binding to the c-Met receptor on the surfaces of epithelial cells, HGF can dissociate epithelial colonies and scatter cells. This activity is thought to be important in the modulation of cancer cell motility and invasion¹⁸⁻²⁰. Matriptase has been shown to activate the latent form of HGF/SF to produce the active growth and motility factor that can bind to and activate the c-Met receptor²¹. In addition, matriptase has been characterized as an in vitro activator of uPA, linking it to the activation of other protease systems important for cancer cell invasion and metastasis²².

In order to test the hypothesis that epithelial-derived cancer cells within a tumor may be a major source of the synthesis and presentation of a protease(s) important for multiple aspects of tumor behavior, including growth and metastasis, we have isolated and characterized a membrane-bound, trypsin-like, serine protease termed matriptase and have identified its integral membrane, Kunitz-type inhibitor, called HAI-1 (hepatocyte growth factor activator inhibitior-1) from T-47D human breast cancer cells and from human milk²³⁻²⁶. Matriptase is a mosaic, transmembrane, trypsin-like serine protease with two potential regulatory modules: two tandem repeats of a CUB (C1r/s, Uegf, and Bone morphogenic protein-1) domain and four tandem repeats of a LDL receptor domain²⁵ (also see updated sequence, Genbank accession #AF118224). This protease is identical in sequence to the protease termed the membrane type serine protease-1, MT-SP1²⁷, and has a high percentage of sequence identity with the mouse serine protease epithin²⁸, the apparent mouse homologue of matriptase. Matriptase was detected in some breast cancer cell lines and in immortalized luminal epithelial cells of the mammary gland, but not in cultured fibroblasts nor in HT1080 fibrosarcoma cells²⁶. Thus, it was proposed that matriptase is produced by epithelial cells in vivo. The matriptase inhibitor, an integral membrane serine protease inhibitor with two Kunitz domains separated by an LDL receptor domain, was initially identified as an inhibitor (HAI-1) of hepatocyte growth factor activator²⁹. This inhibitor is expressed primarily by simple columnar epithelium in multiple human tissues in vivo³⁰.

Matriptase is an epithelial-derived, type 2 integral membrane serine protease. It contains two putative regulatory modules: two tandem repeats of a CUB (C1r/s, Uegf and Bone morphogenetic protein-1) domain, and four tandem repeats of an LDL receptor domain [37]. Matriptase was initially characterized by our group as a major gelatinolytic activity in human breast cancer cells [38,39], and subsequently was purified from human breast milk as a complex with a Kunitz-type serine protease inhibitor, termed hepatocyte growth factor activator inhibitor-1 (HAI-1) [37, 40]. HAI-1 is a type-1, integral membrane, serine protease inhibitor, containing two Kunitz domains and a LDL receptor domain [41]. Matriptase and HAI-1 are co-expressed, both in human mammary epithelial cells and in breast cancer cell lines. Similarly, the expression of both proteins has been detected in human tissue biopsies; a variety of normal epithelial cells and carcinoma cells were positive. Matriptase was also independently cloned from human prostate cancer cells by reverse transcription-PCR, and named membrane-type serine portease-1 (MT-SP-1) [42]. Furthermore, the mouse homologue of matriptase, epithin, was cloned from a thymic stromal-derived subtractive cDNA library; epithin is highly expressed in thymic epithelial nurse cells [43].

The catalytic domain of matriptase contains an Asp residue at the bottom of substrate binding pocket, suggesting that it is a trypsin-like protease. Indeed, matriptase is able to cleave various synthetic substrates containing Arg or Lys at their P1 sites [37, 44]. Three biological substrates have been identified for matriptasse, including urokinase-type plasminogen activator (uPA), hepatocyte growth factor (HGF)/scatter factor (SF), and protease activated receptor-2 (PAR-2) [44,45].

Thus, there remains a need for the elucidation of the mechanisms through which the CEA plays a role in cancer and the design of therapeutic and diagnosis protocols based on the elucidation of those mechanisms.

### SUMMARY OF THE INVENTION

In one embodiment, the present invention provides an in vitro method of diagnosing the presence of a pre-malignant lesion, a malignancy, or other pathologic condition, in a subject, which is characterized by the presence of activated matriptase comprising the steps of:

(A) obtaining a biological sample from a subject that is to be tested for a pre-malignant lesion, a malignancy, or other pathologic condition;

(B) exposing the biological sample to a detectable agent which recognizes and binds to activated matriptase; and

(C) determining whether said detectable agent is bound to the biological sample.

Preferably, the detectable agent is an antibody which specifically binds to activated matriptase. More preferably the antibody is selected from M69 and M123.

In a particular embodiment, the method further comprises exposing the biological sample to one or more antibodies which do not specifically bind to active matriptase., and which preferably bind to the inactive form of matriptase. The ratio between the antibody specifically bound to active matriptase and the total bound antibodies is determined to follow the onset and/or progression of a malignancy.

In another embodiment, the invention provides a method of treating malignancies, pre-malignant conditions, and pathologic conditions in a subject which are characterized by the activated form of matriptase comprising administering a therapeutically effective amount of an agent capable of blocking the activity of active matriptase. Preferably, the matriptase inhibiting agent is an antibody selected from M69 and M123. In another embodiment, the agent is capable of blocking the activation of matriptase by blocking the activity of an agent capable of inducing the activation of matriptase.

In yet another embodiment, the invention provides a method of treating pathologic conditions in a subject which are characterized by the lack of the activated form of matriptase comprising administering a therapeutically effective amount of an agent capable of inducing activation of matriptase. The method is particularly suitable for treatments involving wound healing. Preferably, the agent comprises serum or an extract thereof. In one embodiment, the agent capable of inducing the activation of matriptase is compound comprising a lipid moiety, preferably, the agent comprises lysophosphartidic acid (LPA) or shingosine 1-phosphate (S1P).

### BRIEF DESCRIPTION OF THE DRAWINGS

FIG. 1 Expression analysis of matriptase and HAI-1 in immortalized human breast epithelial, human breast cancer, and ovarian cancer cell lines. Ten micrograms of total RNA were examined for each breast or ovarian cell line by Northern blot analysis using matriptase (A) or HAI-1 (B) specific riboprobes. Analysis of cell lines included two immortalized breast epithelial lines (MCF-10A and A1N4), four ER+ breast cancer cell lines (MCF-7, ZR-75-1, T47D, BT474), nine ER− breast cancer cell lines (SKBR3, MDA-MB-468, -453, -436, -435, -157, and -231, BT549, and Hs578t), and three ovarian cancer cell lines (SKOV3, PA-1, and OVCAR-3). Matriptase expression always correlated with HAI-1 expression, and both were found in 2/2 immortalized breast epithelial cell lines, 4/4 ER+ breast cancer cell lines, 3/9 ER− breast cancer cell lines, and in ⅓ ovarian cancer cell lines.

FIG. 2 Expression analysis of matriptase in mammary tissues. Samples of proteins were extracted using RIPA buffer from normal breast tissue surrounding the breast tumor of three different patients (lanes 13-15) and tumors of ten different patients (lanes 3-12). Proteins (50 μg per lane) were separated by SDS-PAGE, transferred to PVDF membrane, and probed by anti-matriptase mAb 21-9 (Panel A) and anti-HAI-1 mAb M19 (panel B). The positions of matriptase (70-kDa), HAI-1 (55-kDa membrane-bound form and 50-kDa fragment) and the 95-kDa matriptase/HAI-1 complex were indicated according to the samples from the cell-conditioned medium (lanes 1) and the membrane fractions (lanes 2) of T-47D breast cancer cells.

FIG. 3 Expression analysis of matriptase in gynecological tumors. Panels A and B: Samples of proteins (50 μg per lane), which were extracted by RIPA buffer from nine ovarian carcinomas (lanes 3-11) and three stromal-derived tumors, including two fibrothecomas (lanes 12 and 13) and one granulosa cell tumor (lane 14), were analyzed by immunoblot using anti-matriptase mAb 21-9 and anti-HAI-1 mAb M19. The positions of matriptase, HAI-1 and the 95-kDa matriptase/HAI-1 complex were indicated according to the samples from the cell-conditioned medium (lanes 1) and the membrane fractions (lanes 2) of T-47D cells. Panels C and D: Samples of proteins from four uterine carcinomas (lanes 3, 5, 7, and 8) and two patient-matched normal tissues surrounding tumors (lanes 4 and 6) were probed by immunoblot using anti-matriptase mAb 21-9 and anti-HAI-1 mAb M19. The positions of matriptase, HAI-1 and the 95-kDa matriptase/HAI-1 complex are indicated, as described above.

FIG. 4 Expression analysis of matriptase in human colon tumors. Protein samples (50 μg per lane) which were extracted by RIPA buffer from nine colon carcinomas (lanes 3-10) and four normal colon specimens (lane1s 11-14) were examined by western blot using anti-matriptase mAb 21-9 (panel A) and anti-HAI-1 mAb M19 (panel B). The positions of matriptase, HAI-1 and its 95-kDa matriptase/HAI-1 complex were indicated according to the samples from the cell-conditioned medium (lanes 1) and the membrane fractions (lanes 2) of T-47D cells.

FIG. 5 Analysis of matriptase and HAI-1 protein expression in human breast carcinomas by immunohistochemistry. Human breast carcinomas were stained by immunohistochemistry using monoclonal antibodies directed specifically against matriptase (S5) or HAI-1 (M58). Positive staining for matriptase and HAI-1 are observed as a brown precipitate (DAB) within the sections, and nuclei were counterstained with hematoxylin. A metastatic breast adenocarcinoma, shows both cytoplasmic and membranous staining for Matriptase (A, 100X, B, 400X) and HAI-1 (D, 100X, E, 400X) in the breast epithelial cells. No staining is noted in stromal components of the tumor. A colloid breast carcinoma likewise shows a similar staining pattern for matriptase (C, 400X) and HAI-1 (F, 400X).

FIG. 6 Analysis of matriptase and HAI-1 protein expression in normal and hyperplastic human breast epithelium by immunohistochemistry. Intense staining for matriptase is seen in the duct and mild or no staining in the surrounding terminal duct lobular units (TDLU) of this area of normal breast epithelium surrounding a breast carcinoma (Panel A, 20X). A duct with usual ductal hyperplasia shows intense staining for matriptase, and the surrounding TDLUs show mild staining (Panel B, 40X). Focal staining for HAI-1 is noted in the TDLU, and no staining is seen in the surrounding duct (Panel C, 40X). A high power view of the same lobule shows preferential staining of the lobular cells, and no staining is seen in the myoepithelial cells (Panel D, 200X).

FIG. 7 Analysis of the expression of matriptase in invasive primary breast tumors by in situ hybridization. A matriptase-specific anti-sense probe (A and C) and the corresponding control sense probe (B and D, respectively) were hybridized to paraffin-embedded sections of primary breast tumors as described in Materials and Methods. The matriptase anti-sense probe shows reactivity with the cancer cells within the sections and lack of reactivity with stromal elements such as fibroblasts and adipocytes (A and C). Control sense probes do not show any reactivity with the breast cancer sections (B and D), demonstrating the specificity of the labeled anti-sense probe.

FIG. 8 The tissue concentration of matriptase in human breast tumors and the surrounding tissues. The tissue concentration of matriptase was determined by immunoblot. The concentration of a purified matriptase standard was determined by comparison with a BSA standard curve resolved by SDS-PAGE and stained with Coomassie Blue. Different amounts of purified matriptase were then used (80 pg, 200 pg. 400 pg, and 800 pg lanes 1-4) to generate a standard curve for the immunoblot. Protein samples (50 μg) from six human breast tumors (lanes 5-10) and two surrounding tissues (lanes 11 and 12) were examined by immunoblot and compared to the standard curve for matriptase. The final concentration of each specimens was calculated and indicated.

FIG. 9 (Table I) Comparison of the expression of matriptase and HAI-1 with markers of epithelial differentiation and in vitro invasiveness. The expression of matriptase and HAI-1 in immortalized human breast epithelial cell lines and cancer cell lines as determined in FIG. 1 are compared with the expression of E-cadherin and zona occludens-1 (ZO-1)—markers typical of an epithelial differentiation, and vimentin—a marker typical of mesenchymal differentiation, as determined by others³⁴⁻³⁶. Matriptase and HAI-1 are expressed in the same cells which express either E-cadherin or ZO-1, or which lack expression of vimentin. ER=estrogen receptor status; NA=data not available; *in vitro invasiveness as assessed in vitro by invasion into the extracellular matrix preparation Matrigel, as determined by Sommers, et al³⁴.

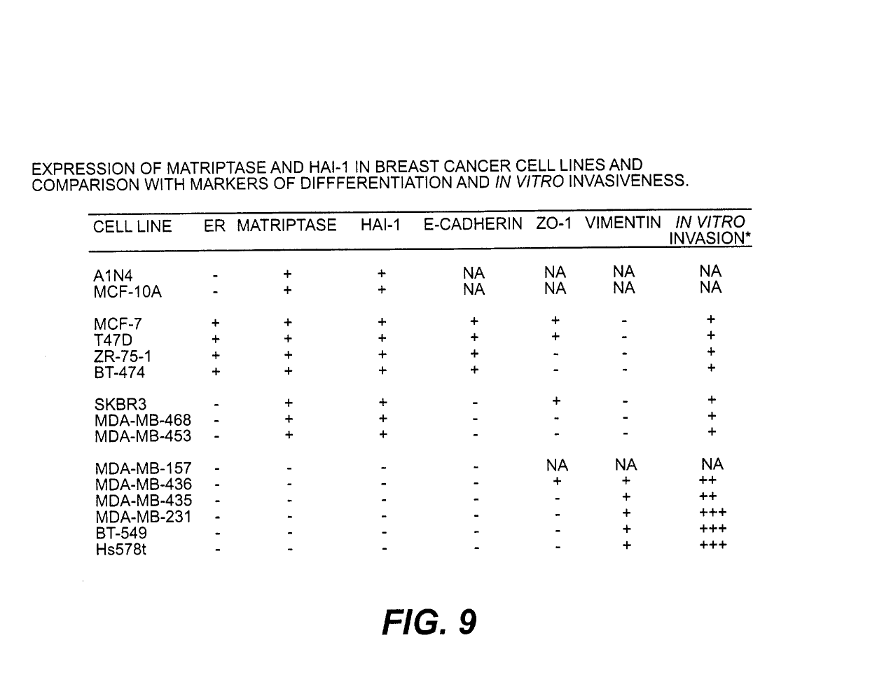

FIG. 10 Non-reduced/reduced diagonal gel electrophoresis of complexed and uncomplexed matriptases. Matriptase was purified by immunoaffinity chromatography using anti-matriptase mAb 21-9 from conditioned medium of T-47D human breast cancer cells and from human milk. These samples were treated with SDS sample buffer in the absence reducing agents, incubated at 95° C. for 5 min, and then resolved by SDS-PAGE (1^{st}-D boiled). Under boiled, non-reduced conditions, the 95-kDa complexed matriptase preparation from human milk was converted to the 70-kDa matriptase and the 40-kDa fragment doublet of HAI-1 (panel A 1^{st}-D). A non-characterized co-purified protein, observed between matriptase and the HAI-1 fragment, was also seen in this preparation. In the preparation from T-47D cells, only the uncomplexed form of matriptase was purified; no HAI-1 was co-purified (panel B, 1^{st}-D). Parallel gel strips were sliced, boiled in 1× SDS sample buffer in the presence of reducing agents for 5 min, placed on a second SDS gel, and electrophoresed. After these procedures, complexed matriptase (panel A) was dissociated into two components with apparent sizes of 45-kDa (A chain) and 25-kDa (B chain). However, uncomplexed matriptase (panel A) remained as a single chain.

FIG. 11 Inhibition of matriptase by HAI-1. Matriptase and HAI-1 were isolated from human milk by anti-matriptase mAb 21-9 immunoaffinity chromatography, as described previously [40], and were maintained in an uncomplexed status in elution buffer, 0.1 M glycine, pH 2.4. To demonstrate binding to and inhibition of matriptase by HAI-1, this preparation was brought to pH 8.0, incubated at 37° C. for 0, 5, 30, and 60 min, and subjected to immunoblot using anti-matriptase mAb 21-9 (panel A), gelatin zymography (panel B), and to a cleavage rate assay using the synthetic, fluorescent substrate, BOC-Gln-Ala-Agr-7-amido 4-methylcoumarin (panel C). A milk-derived, matriptase-related 110-kDa protease which does not form a complex with matriptase and HAI-1, is also detected by immunoblotting (panel A) [40].

FIG. 12 Production of mAbs directed against the two-chain form of matriptase. Anti-matriptase mAbs, produced in our previous study [40], were subjected to further selection for their differential immunoreactivity against the purified, two-chain form (lanes 1, 3, and 5) or the purified single-chain form (lanes 2, 4, and 6) of matriptase. The majority of these anti-matriptase mAbs, as represented by M32, showed immunoreactivity against both the two-chain and the single-chain forms of matriptase (lanes 1 and 2). In contrast, mAbs M123 and M69 only recognized the two-chain form of matriptase (lanes 3 and 5), but not the single-chain form (lanes 4 and 6). It was noticed that the two-chain form of matriptase has a slightly slower migration rate on SDS-PAGE than the single-chain form.

FIG. 13 Transient activation of matriptase by serum. A1N4 cells were maintained for 2 days in low serum. Cells were then stimulated with 0.5% FBS in IMEM (Panels A and B) for the indicated times. The cells were harvested, and expression of two-chain matriptase, total matriptase, and HAI-1 were analyzed by immunoblot using mAbs M69, M32, and M19, respectively. In panel C, serum-starved cells were exposed to either 0.5% or 5% FBS for 16 hrs, and expression of two-chain matriptase was determined by western blotting.

FIG. 14 Immunofluorescence analysis of actived matriptase, following serum stimulation of A1N4 cells. A1N4 cells were stimulated with IMEM (a,c) or IMEM containing 1% serum (b,d,e) for 40 min. Non-permebilized cells were incubated with mAb M69 to detect the two-chain form of matriptase (a,b), with mAb M32 to detect total matriptase (c,d), or with FITC-labeled secondary antibody alone (e).

FIG. 15 Matriptase activation is induced by sera from various animal species. A1N4 cells were maintained for 2 days in medium supplemented with 0.5% FBS, were stimulated for 1 hr with 1% sera from the indicated species. Total cell lysates were analyzed by western blotting for the presence of two-chain form matriptase and total matriptase using M69 and M32 antibodies, respectively.

FIG. 16 Activation of matriptase is accompanied by the shedding of matriptase from cells. To investigate the release of matriptase into medium following the activation of matriptase, the conditioned media were collected at the indicated times, following the addition of 0.5% FBS to the cells. These media were concentrated and examined by western blotting for the expression of the two-chain matriptase and total matriptase, using mAb M69 and M32, respectively.

FIG. 17 Expression of activated Matriptase in primary human breast carcinoma. A five micron section from a formalin-fixed human breast carcinoma was stained using the monoclonal antibody M69 that recognizes the two-chain form of the enzyme (panel A) or a control mouse IgG (panel B). The M69 antibody stains the carcinoma cells within the tumor, but not the fibroblasts in the stroma.

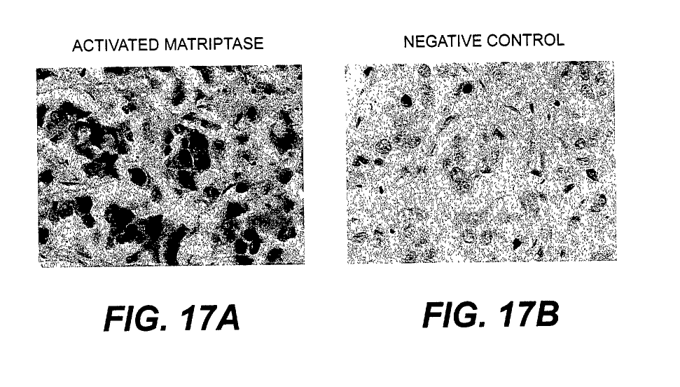

FIG. 18 Expression of Matriptase in peripheral blood mononuclear cells. The expression of Matriptase in peripheral blood mononuclear cells (PBMCs) was determined by FACs analysis using the Matriptase-specific monoclonal antibody M32. PBMCs were obtained from whole blood by centrifugation on a Ficoll density gradient, and collection of the buffy coat containing PBMCs. After washing in PBS, cells were stained with the Matriptase-specific monoclonal antibody M32 and a phycoerytherin conjugated anti-mouse secondary antibody and fixed prior to FACS analysis. Lymphocyte (top traces) and granulocyte (bottom traces) populations were separated by forward and side scatter and the intensity of Matriptase-specific fluorescence was determined for unstained cells (blue), cells stained with secondary antibody alone (green), and for cells stained with the Matriptase antibody M32 (red). Results show the presence of intense Matriptase staining in the granulocyte population, indicating the presence of this protease in a leukocyte subpopulation important in host immune responses.

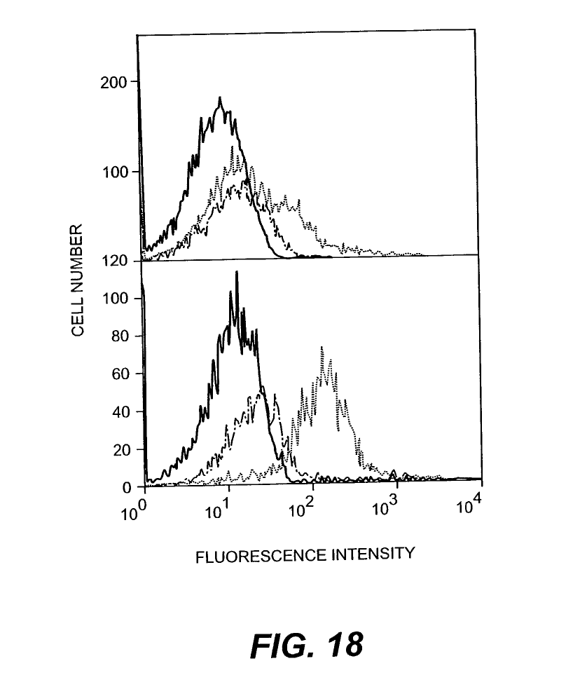

FIG. 19 Expression of matriptase by vesicular smooth muscle cells in vivo. Sections of paraffin embedded human lymph node from breast cancer patient were stained with anti matriptase mAb S5 (panel A) and mouse IgG (panel B). Matriptase was clear stained on the smooth muscle cells (panel A).

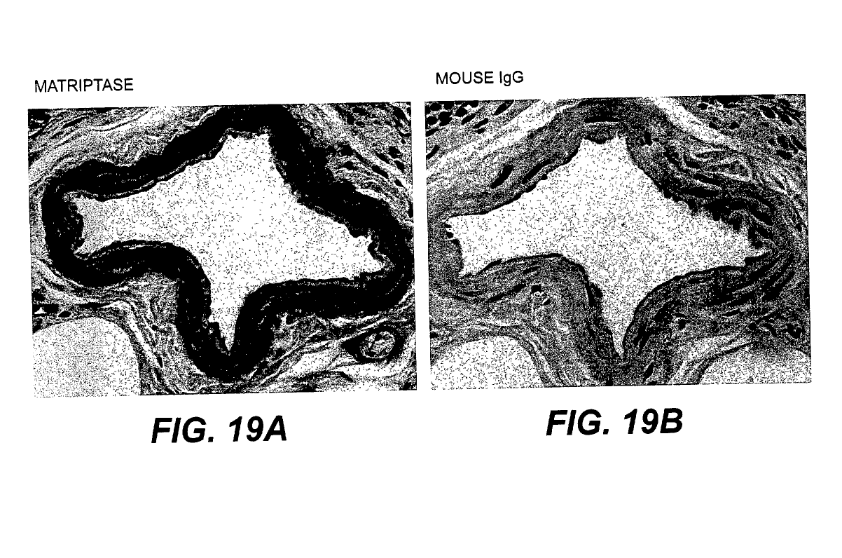

FIG. 20 Size fractionation of the serum-derived inducer of matriptase activation.

FIG. 21 Lipoproteins induce the activation of matriptase.

FIG. 22 LPA and S1P induce the activation of matriptase.

FIG. 23 Immunofluorescence of actin rearrangement and activated matriptase following LPA and SIP stimulation.

FIG. 24 (Table 2).

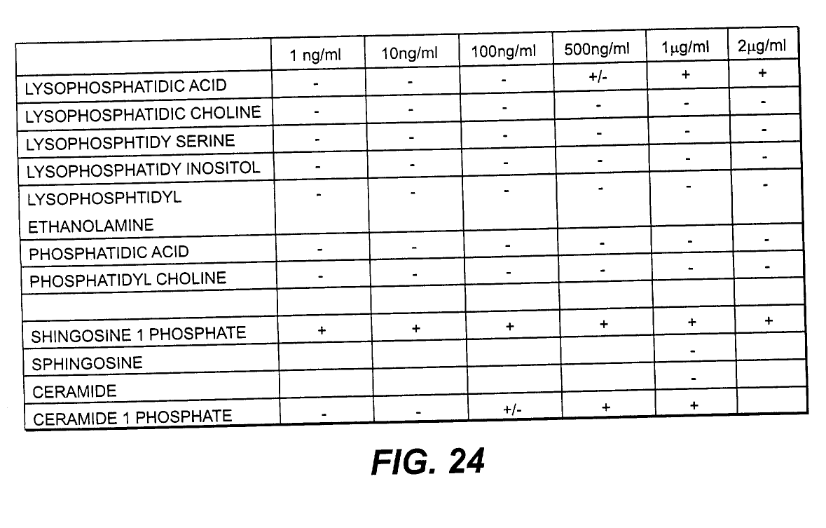

### DETAILED DESCRIPTION OF THE PREFERRED EMBODIMENTS

The present invention is based, in part, on the characterization of the expression of matriptase in human primary breast cancer and in human breast cancer cell lines. Data from the breast cancer cell lines suggest that the expression of matriptase correlates with markers of an epithelial phenotype. Results from primary tumors indicate that the protease is expressed by tumors of epithelial origin. Matriptase is expressed predominately by the epithelial elements of carcinomas and not by fibroblasts. The in vivo epithelial origin of matriptase and its expression by breast cancer cells highlight the potential role of matriptase in the activation of latent growth factors and proteases at the breast epithelial cell surface and in the development, growth, invasion, and metastasis.

Matriptase thus potentially serves as an epithelial-derived, membrane-bound activator for another secreted protease, for a growth factor, and for a cell surface G-protein-coupled receptor. Considering its potent, trypsin-like activity and its potential role in the activation of other important biomolecules, we hypothesized that matriptase activity is likely to be tightly regulated. In the current study, we have explored the mechanisms involved in the regulation of the proteolytic activity of matriptase in normal human mammary epithelial cells.

I. Expression of Matriptase and its Cognate Inhibitor HAI-1 by Normal and Malignant Epithelial Cells In Vitro and In Vivo

Materials and Methods

Cell lines and culture conditions—All breast and ovarian cancer cell lines were obtained from the Lombardi Cancer Center Tissue Culture Shared Resources. Cells were maintained in culture by growth in Iscove's Minimal Essential Media (IMEM, Gibco BRL, Rockville, Md.) supplemented with 5% fetal bovine serum (FBS) at 37° C. and 5% CO₂, with the exception of the MCF-10A cell line, grown in DMEM/HAM F-12 (Gibco, BRL, Rockville, Md.) supplemented with 0.5% FBS, 0.5 mg/mL hydrocortisone, 10 μg/mL insulin, and 10 ng/mL EGF, and the 184 A1N4 human mammary epithelial cell line, grown in IMEM supplemented with 5% FBS, 0.5 mg/mL hydrocortisone, 5 μg/mL insulin, and 20 ng/mL EGF.

Monoclonal antibodies—Anti-matriptase and anti-HAI-1 mAbs were prepared as described previously^{24;26}. Immunohistochemistry-competent anti-matriptase mAb S5 (IgG1) was prepared by hybridoma technology using formalin-treated matriptase, isolated from T-47D cells, as immunogen. A panel of mAbs was selected by its ability to stain paraffin-embedded breast cancer sections. Monoclonal Ab S5 was selected from these mAbs for its ability to recognize matriptase by immunoblot.

Extraction of proteins from frozen human tumors—Frozen human tumors from various sites were obtained from the Histopathology and Tissue Shared Resource at the Lombardi Cancer Center, Georgetown University. The tumor specimens were kept frozen with liquid nitrogen and ground to a fine powder by mortar and pestle. The specimens were extracted using RIPA buffer (150 mM NaCl, 1% NP-40, 0.5% deoxycholic acid, 0.1% SDS, and 50 mM Tris pH 8.0). The insoluble debris was removed by centrifugation, and the protein concentration was determined by BCA protein assay (Pierce, Rockdord, Ill.).

Western blotting—Proteins were resolved by 10% SDS-PAGE, transferred overnight to polyvinylidene fluoride (PVDF), and subsequently probed with mAbs as indicated. Immunoreactive polypeptides were visualized using HRP-labeled secondary antibodies and the ECL detection system (NEN, Boston, Mass.).

Northern blotting—Total RNA was extracted from cell lines with RNAzol reagent (Tel-Test, Inc., Friendswood, Tex.) according to the manufacturer's instructions. Ten micrograms of total RNA from each cell line was resolved by electrophoresis on a 1.2% phosphate-buffered agarose gel containing 2M formaldehyde. RNA was subsequently transferred to hybond-N nylon membranes (Amersham Pharmacia Biotech, Buckinghamshire, England) and hybridized to ³²P-labeled riboprobes at 65° C. for 36 hours, followed by three washes in 0.1×SSC/0.1%SDS at 80° C. for 30 minutes each to remove unbound probe. To generate the labeled riboprobes, the coding sequences of Matriptase and HAI-1 were cloned into the pcDNA3.1 vector (Invitrogen, San Diego, Calif.), and were linearized with an appropriate restriction enzyme just 5′ of the coding sequences. These linearized vectors were used in in vitro SP6 RNA polymerase reactions with ³²P-UTP (3000 Ci/mmol, NEN, Boston, Mass.) to generate labeled antisense riboprobes. To control for approximately equal loading of RNA from each cell line, labeled riboprobes directed against the message of the ribosomal protein 36B4 were generated in a similar fashion, except that a vector containing approximately 500 bp of the coding sequence was used. Signals on hybridized membranes were visualized by exposure to X-OMAT AR imaging film (Eastman-Kodak, Rochester, N.Y.) for 4 to 12 hours at −80° C.

Immunohistochemistry—Paraffin-embedded sections of human primary breast cancers were obtained from the Histopathology and Tissue Shared Resource at the Lombardi Cancer Center, Georgetown University. Briefly, 5 tumor sections were heated in an oven to 56° C. for 3 hours and then de-waxed in xylene. Slides were then rehydrated by immersion in a decreasing gradient of ethanol in water. Endogenous peroxidase activity was quenched by immersion in 1.5% H₂O₂/methanol for 10 minutes, followed by washes in water and PBS. Sections were blocked for 30 minutes in blocking buffer (2% goat serum/5% BSA in PBS) prior to incubation with the primary antibody. Sections were incubated in the presence of the matriptase-specific monoclonal antibody clone S5 (IgG1) at a concentration of 1 μg/mL, the HAI-1 specific monoclonal antibody clone M58 (IgG1) at a concentration of 5 μg/mL or mouse IgG 1 at a concentration of 5 μg/mL in blocking buffer for 1 hour at RT. After incubation in the primary antibody, sections were washed in PBS to remove unbound antibody, and then were incubated with a biotinylated goat anti-mouse secondary antibody. After washes in PBS, the staining was completed by incubation with strepavidin-HRP and DAB colorimetric reagents from the BioGenex immunohistochemistry kit (San Ramon, Calif.) according to the manufacturer's protocol. As a control, non-relevant mouse IgG1 was used in place of the specific monoclonal antibodies at the equivalent dilutions. The colorimetric reaction for the control slides was developed for the same amount of time as the experimental slides, and did not show any development of the color reagent.

In situ hybridization—Probes for use in in situ hybridization were prepared by generatating digoxigenin-labeled sense and anti-sense RNA riboprobes using the Dig-RNA labeling kit (Boehringer-Mannheim, Mannheim, Germany) according to a modified maufacturer's protocol. Briefly, a 650 bp BamHI-SacII fragment of the Matriptase sequence corresponding to the 5′ end of the Matriptase cDNA was cloned into the pBluescript SK-vector (Stratagene, La Jolla, Calif.). This vector was subsequently linearized with SacII or BamHI and used as a template for the synthesis of sense and antisense digoxigenin-labeled riboprobes, respectively, with T7 or T3 RNA polymerase (Gibco BRL, Rockville, Md.), according to the manufacturer's protocol, using digoxigenin-11-UTP. Synthesized probes were purified by G50 column chromatography to remove unincorporated nucleotides, including unincorporated digoxigenin-11-UTP. The concentration of the labeled riboprobes was determined spectrophotometrically. The accuracy of the concentration assignment was confirmed by analysis of the riboprobes by 1% agarose/2M formaldehyde gel electrophoresis, followed by ethidium bromide staining. The equal efficiency of digoxigenin incorporation into sense and antisense probes was confirmed by dot-blotting of equal amounts of probe onto hybond-N nylon membranes, followed by detection of labeled riboprobe with an alkaline phosphatase conjugated anti-digoxigenin antibody and colorimetric substrate (not shown). In addition, the efficiency of digoxigenin incorporation was confirmed by dot blotting equal amounts of denatured double-stranded vector containing the full-length sequence of Matriptase and probing these blots with digoxigenin-labeled sense or antisense probes for Matriptase. Equal signals were observed for equal amounts of sense or antisense probe used in the hybridization to membrane-bound plasmid (not shown). For detection of Matriptase mRNA in paraffin-embedded breast cancer sections, 20 ng of labeled sense or antisense riboprobe was used in a standard protocol provided by Boehringer Mannheim (Mannheim, Germany). Briefly, 5 μm paraffin-embedded breast cancer tissue sections were de-paraffinized, rehydrated, treated with 0.2M HCl, permeabilized with proteinase K, and post-fixed with 4% paraformaldeyde prior to pre-hybridization in 50% foramide/1×SSC at 65° C. and hybridization at 65° C. in hybridization buffer for 12 hours in a humidified chamber. After hybridization, unbound probe was removed by two washes in 2×SSC, two washes in 1×SSC, and two washes in 0.1×SSC at 42° C. Bound probe was detected by use of an alkaline-phosphatase conjugated anti-digoxigenin antibody that produces an insoluble blue precipitate in the presence of a nitrotetrazolium blue/X-phosphate color solution. Sense and anti-sense probes were hybridized and washed under identical conditions, and the colorimetric reactions were stopped at the same time for sense and anti-sense hybridized sections.

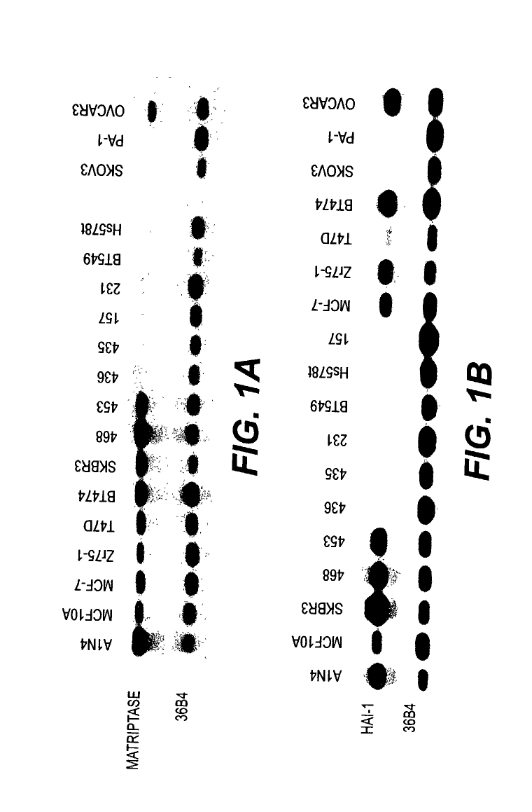

In vitro expression of matriptase and HAI-1 correlates with the expression of epithelial markers in breast cancer cell lines—In order to determine the expression of Matriptase and HAI-1 in breast cancer cell lines, Northern blotting was performed using RNA from a panel of breast cancer cell lines (FIG. 1). Expression of matriptase and HAI-1 in human breast cancer cell lines was completely concordant; matriptase and HAI-1 were found in 4/4 estrogen receptor positive (ER+) and 3/9 estrogen receptor negative (ER−) breast cancer cell lines. Both were expressed in 2/2 ER-negative immortalized breast epithelial cell lines tested. Neither were expressed by a primary breast fibroblast cell line (data not shown). Matriptase and HAI-1 were detected in ⅓ human ovarian cancer cell lines tested (FIG. 1). The expression of matriptase and HAI-1, or lack thereof, by all of the cell lines was confirmed at the protein level by western blot analysis (data not shown).

When the expression of Matriptase and HAI-1 were compared with markers of an epithelial morphology (E-cadherin, ZO-1) or of a generally mesenchymal morphology (vimentin), the expression of the two proteins correlated with epithelial cell markers (table 1) and never with vimentin. The protease and inhibitor were found in all ER+ cell lines and in a smaller number of ER− cell lines tested. However, this trend towards expression in ER+ tumor cells and absence in ER− cells was not observed in our studies of primary breast tumors (see below). In primary breast tumors, the expression of both have been found in both ER+ and ER− tumors, with no trend toward either an ER+ or ER− status. Matriptase and HAI-1 do not appear to be regulated at the transcriptional level by estrogen or progesterone, as no change in mRNA levels for matriptase or HAI-1 were observed in estrogen-depleted MCF-7 cells when treated with 100 nM 17β-estradiol or with 100 nM 17β-estradiol plus 100 nM progesterone (data not shown).

Expression analyses of matriptase and HAI-1 by immunoblot analysis of human primary tumors—The correlation between matriptase/HAI-1 expression and epithelial markers in cultured cells suggests that the matriptase/HAI-1 system could also be expressed in vivo by normal epithelial cells and by epithelium-derived cancer cells. To test this hypothesis, we examined various epithelium-derived and non-epithelium-derived frozen human tumor specimens and the surrounding tissues of these tumors by protein immunoblotting. The epithelium-derived tumors included 10 breast, 9 ovarian, 4 uterine, and 8 colon carcinomas. Non-cancerous tissues surrounding these carcinomas were also included in the lysates tested. Non-epithelial tumors included three stroma-derived ovarian tumors and ten sarcomas with various origins and histological grades.

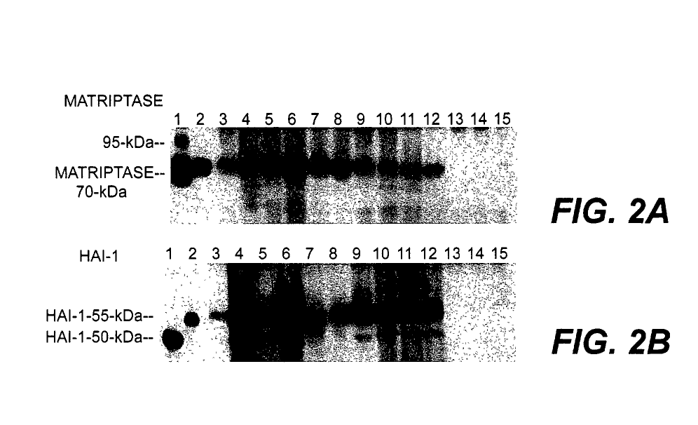

Breast tumors—Expression of matriptase in ten infiltrating human breast carcinomas (nine ductal carcinomas and one colloidal carcinoma; five were estrogen receptor positive specimens and five estrogen receptor negative) was examined by western blot and compared to three samples of non-cancerous breast tissue surrounding a tumor. In these surrounding normal tissues, matriptase was detected at very low levels or below the detection sensitivity (FIG. 2A lanes 13 to 15). In contrast, higher levels of expression of matriptase was observed in all ten of the primary breast carcinomas examined (FIG. 2A lanes 3-12), consistent with the higher epithelial cell-derived component of these specimens. The expression of HAI-1 was also observed in these ten human breast specimens, but under the detection sensitivity by western blot in the surrounding non-cancerous tissue, which is composed primarily of stromal (non-epithelial) elements. Expression of HAI-1 fluctuated widely among these specimens (FIG. 2B).

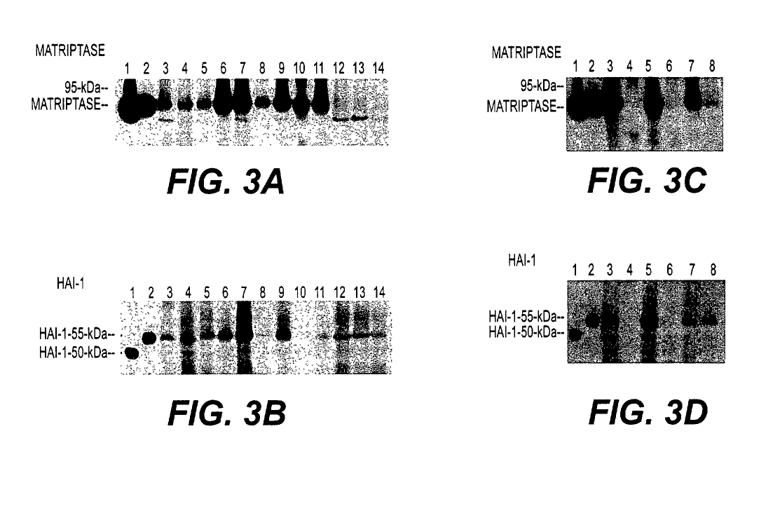

Gynecological tumors—We further investigated the expression of matriptase in gynecological tumors (FIG. 3). There are more than 25 major types of ovarian neoplasms. These are classified into three groups based on cell of origin: those of the germinal surface epithelium, the gonadal stroma, and germ cells. We examined ovarian tumors both of epithelial origin and of stromal origin. Among the nine tumors of epithelial origin, matriptase was detected at moderate to high levels (FIG. 3A lanes 3-11). In contrast, matriptase was not detected in three sex cord/stromal tumors, including a granulosa cell tumor (FIG. 3A lane 14), and two fibrothecomas (FIG. 3A lanes 11 and 12). The negative results in sex cord/stromal tumors again suggest that expression of matriptase is restricted to tumors of an epithelial origin. Expression of HAI-1 varied widely among these ovarian carcinomas (FIG. 3B lanes 3-11). In one of these specimens, HAI-1 was below the detection sensitivity (FIG. 3B lane 10), whereas matriptase was detected at a high level (FIG. 3A lane 10). HAI-1 was not detected in the three sex cord/stromal tumors. A minor, non-specific band with a migration similar to that of HAI-1 could not be depleted with anti-HAI-1 monoclonal antibody; this band was also observed in both matriptase and HAI-1 western blots (FIGS. 3A and B lanes 12-14).

We also examined the expression of matriptase and HAI-1 in four uterine carcinomas (FIGS. 3C and D lanes 3, 5, 7, and 8) and in two patient-matched normal tissues (FIGS. 3C and D lanes 4 and 6). Expression of matriptase was observed strongly in three out of four (FIG. 3C lanes 3, 5, and 7), and weakly in one out of four (FIG. 3C, lane 8) cancer specimens, while the two normal tissues were below the detection limit (FIG. 3C lanes 4 and 6). HAI-1 expression was observed at a high level in one specimen (FIG. 3D lane 5) and at low levels in the other three specimens (FIG. 3C lanes 3, 7, and 8).

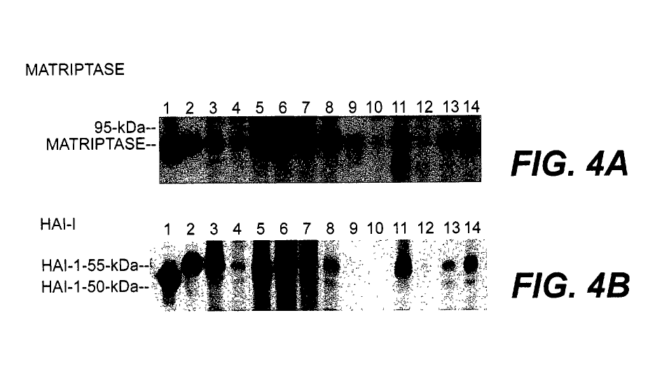

Colon tumors—Eight colon carcinoma specimens (FIG. 4A lanes 3-10) and five normal colon specimens (FIG. 4A lanes 11-14) were also examined. Expression of matriptase fluctuated among colon carcinomas as well as among normal colon tissues. In contrast to breast and gynecologic carcinomas, expression of matriptase in some normal colon tissues was as high as that seen in cancer specimens, consistent with the high percentage of epithelial cells present in normal colon tissues relative to that of normal breast and ovarian tissue. Expression of HAI-1 also varied among these specimens (FIG. 4B), but tended to correlate with matriptase expression.

Sarcomas—Ten human sarcomas were examined for the expression of matriptase (data not shown). These included three high grade osteosarcomas, three well differentiated (low grade) liposarcomas, two malignant fibrous histiocytomas which were clinically metastatic, one dermatofibrosarcoma protuberance (a low grade sarcoma), and one high grade leiomyosarcoma which was most likely of uterine origin. Matriptase was below the detection limit or barely detectable for all of the sarcomas. HAI-1 was below the detection limit for all ten sarcomas.

From this preliminary screening, matriptase was detected in all 31 human carcinomas tested. In contrast, expression of matriptase and HAI-1 was negligible or not detected in all of the 13 non-epithelial tumors tested. These results suggest that matriptase is selectively expressed in epithelium-derived tumors in vivo, consistent with the observation that matriptase was detected in cultured cells that tended to express epithelial makers, but not in cells with a mesenchymal maker. Matriptase and HAI-1 are found at higher levels in breast and gynecologic cancer tissue when compared to normal surrounding tissue; however, this is likely due to the increased epithelial cellularity of cancer tissue versus normal tissue. This observation is supported by the fact that matriptase was detected at high levels in some normal colon tissues for which the epithelial element represents a major portion of normal colon tissue.

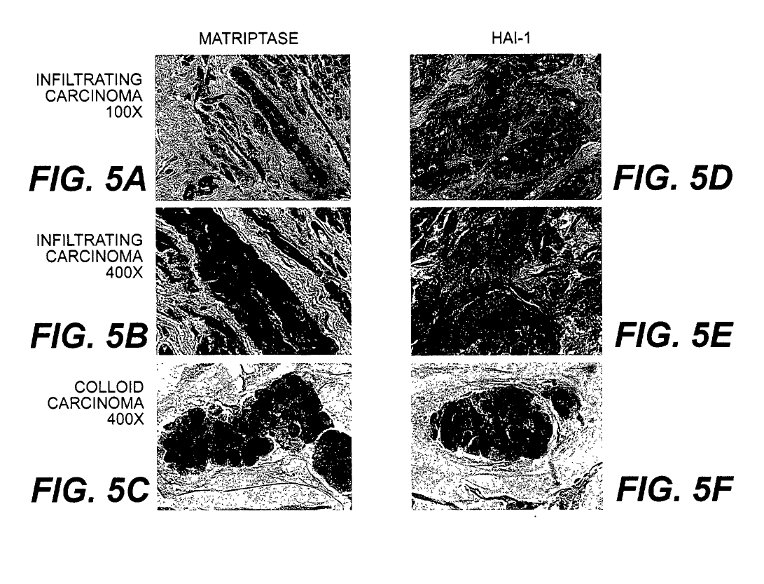

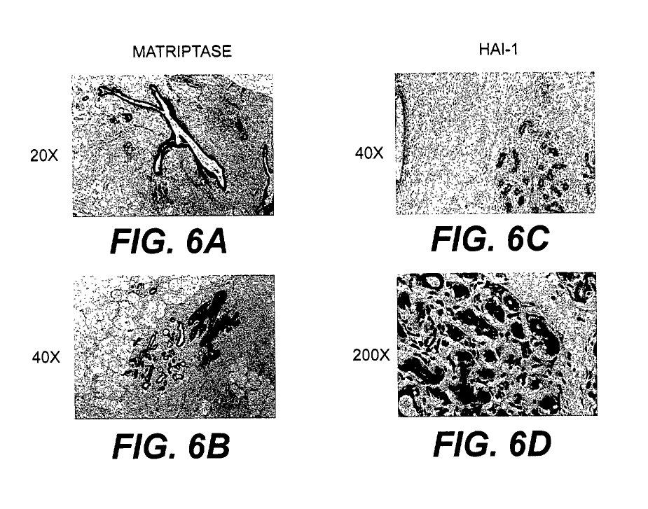

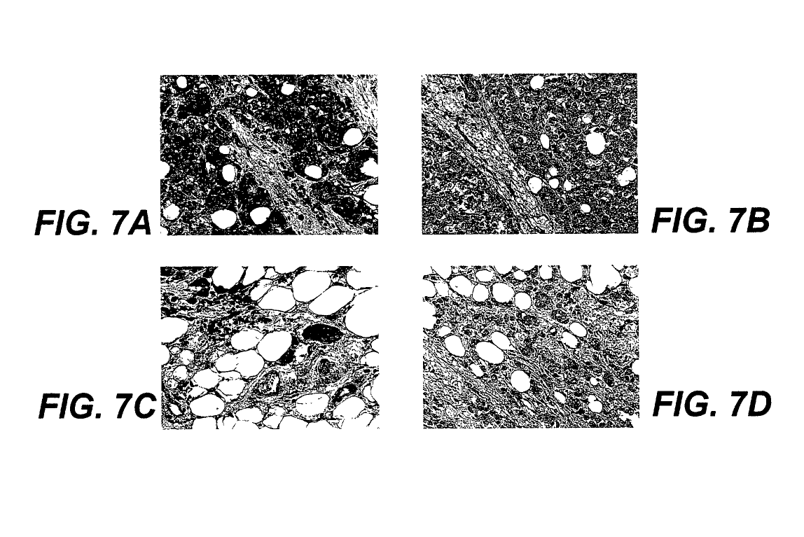

Matriptase protein and mRNA are detected in normal and cancerous epithelial cells in human breast tumor sections—To further determine which cell types express matriptase protein and mRNA in primary tumor specimens, immunohistochemstry and in situ hybridization using digoxigenin labeled riboprobes were performed using formalin-fixed, paraffin-embedded human breast carcinomas. Matriptase protein was detected in breast cancer cells (FIGS. 5A, B, and C) as well as in surrounding normal breast epithelial cells with comparable intensity (FIGS. 6A and B). Within normal breast epithelium and in surrounding hyperplastic ducts, the ducts stained intensely, while mild or no staining was observed in surrounding terminal duct lobular units (TDLU). The localization of matriptase to the breast cancer cell component of the breast tumors was confirmed by in situ hybridization using digoxigenin-labeled riboprobes (FIG. 7). The localization of HAI-1 protein was also determined by immunohistochemistry in the primary breast tumors and in surrounding normal breast tissue. The inhibitor co-localized with that of matriptase in the tumor cell compartment (FIGS. 5D, E, and F). Within surrounding normal breast tissue, focal staining was observed for HAI-1 in the TDLUs, with no staining seen in myoepithelial cells (FIGS. 6C and D). Normal ducts surrounding TDLUs show variable staining for HAI-1. These results are consistent with the expression of HAI-1 by epithelial elements of breast and other tissues as found by others³⁰.

The subcellular localization of the immunohistochemical staining for matriptase and for HAI-1 was observed in both the cytoplasm and at the cell membrane. The latter observation is consistent with the fact that the two are integral membrane proteins, and the former may be explained by the internalization of the proteins or the synthetic pool of these molelcules. These results are consistent with the localization of matriptase at the cell surface and in the cytoplasm of cultured breast cancer cells stained by immunofluorescent techniques²³.

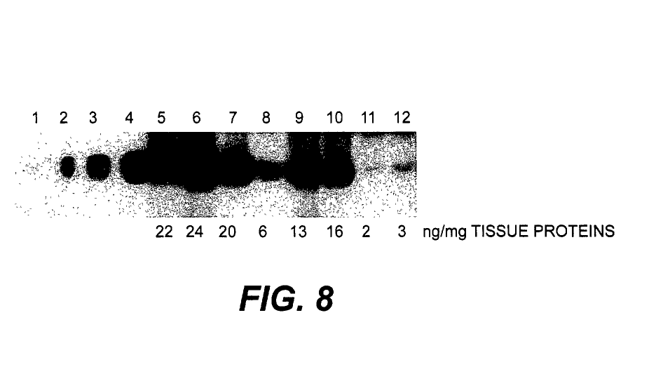

Tissue concentration of matriptase in breast carcinomas—The tissue concentrations of matriptase in six breast carcinomas and two surrounding non-cancerous breast tissues were determined by immunoblot (FIG. 8). The concentration of matriptase in the six carcinomas ranged from 13 to 24 ng/mg tissue protein, with the exception of a colloidal carcinoma specimen, which contained 7 ng/mg tissue protein. The relatively low matriptase protein level in the colloidal carcinoma may be explained by the high proportion of non-epithelial elements in this tumor type. The concentration of matriptase in the two normal surrounding breast tissue specimens was 2 and 3 ng/mg tissue protein, respectively. Again, this lower value in normal breast tissue is consistent with the lower epithelial representation of this tissue relative to breast carcinomas.

Matriptase is a mosaic, transmembrane serine protease isolated from human breast milk and initially identified in human breast cancer cell conditioned media by gelatin zymography²². Matriptase is identical to the membrane-type serine protease-1 (MT-SP1), and is likely to be the human homologue of the mouse serine protease epithin based upon its high degree of sequence identity and syntenic chromosomal location (human chromosome 11 and mouse chromosome 9)^{28;31}. The purified serine protease domain of MT-SP1 has recently been shown to cleave and activate the urokinase plasminogen activator and the protease-activated receptor-2 (PAR-2)²². Active uPA cleaves and activates the serine protease plasmin, and this may lead to degradation of the extracellular matrix and activation of other protease systems involved in the spread of cancer cells, such as MMP-2 and MMP-9³². In addition, matriptase can cleave hepatocyte growth factor/scatter factor (HGF/SF) to an active form able to activate the c-met receptor and induce cell scattering²¹. Many studies have implicated HGF in the growth and motility of various cell types, as well as in the angiogenesis and growth of tumors³³. Therefore, this protease may play a role in the growth and/or invasion of human breast cancer via its activation of pro-uPA and pro-HGF. To further characterize the expression of matriptase, and its cognate inhibitor HAI-1, in breast cancer cell lines in vitro and in human primary breast cancers and other cancers and normal tissues in vivo, we have expanded our expression analysis of matriptase in this study.

Matriptase was initially identified by gelatin zymography only in ER+ hormone-dependent breast cancer cells, including T-47D, MCF-7, and ZR-75-1 and BT474²³, but not in the ER− homone-independent cell lines MDA-MB-231, MDA-MB-435, MDA-MB-436, and BT-549²³. To test the hypothesis that matriptase may be expressed exclusively by ER+ breast cancer cell lines, we screened additional ER+ and ER− cell lines for expression of matriptase and HAI-1 by western and Northern blotting. We found that both matriptase and HAI-1 protein and mRNA are expressed in three ER− cell lines, SKBR3, MDA-MB-453, and MDA-MB-468, but not in numerous other ER− cell lines. Thus, the expression of matriptase and HAI-1 does not always correlate with the expression of the estrogen receptor in cultured breast cancer cells. The expression of Matriptase and HAI-1 in vivo in primary breast tumors differs from the expression pattern in cultured cells in that all ER-tumors examined to date have been found to express matriptase and HAI-1, as have all ER+ tumors. Therefore, matriptase and HAI-1 expression does not seem to rely on the expression status of the estrogen receptor either in vitro or in vivo. Furthermore, both genes do not appear to be transcriptionally regulated by estrogen or progesterone, as neither is induced by the exposure of estrogen-stripped MCF-7 cells to 17β-estradiol nor to 17β-estradiol plus progesterone.

The expression of matriptase did correlate with the expression of markers of an epithelial phenotype (E-cadherin or ZO-1 positive) and did not correlate with the expression of a mesenchymal phenotype (vimentin positive). The co-expression of matriptase and HAI-1 negatively correlates with the previously determined in vitro invasion of these cells into matrigel³⁴. This data may be interpreted as indicating that matriptase is not involved in augmenting the invasive phenotype of cultured breast cancer cells. However, the significance of this observation with regard to invasion in vivo in human carcinomas is unclear, since most of the breast cancer cell lines tested originated from pleural effusions or ascites in human cancer patients with metastatic disease, and are therefore by definition invasive. Additionally, all of the cell lines tested have been cultured for many passages, allowing significant opportunity for phenotypic and genetic drift. Furthermore, we found the expression of matriptase and HAI-1 in 10/10 primary invasive breast cancers examined. This observation and the fact that primary invasive breast cancers rarely express mesenchymal markers such as vimentin further suggests that the lack of expression of matriptase and HAI-1 in vitro in a subset of invasive breast cancer cell lines may be a tissue culture artifact.

While no absolute correlation exists between matriptase/HAI-1 expression and estrogen receptor expression in cultured breast cancer cells, a reverse correlation between matriptase/HAI-1 and vimentin, a mesenchymal marker, was observed among these breast cancer cells. This tendency towards lack of expression in cells that express a mesenchymal phenotype is consistent with our previous study that matriptase was not detected in cultured human fibroblasts and HT-1080 fibrosarcoma cells²⁶. The in vivo expression analyses for matriptase and HAI-1 appear to support this in vitro correlation. Expression of matriptase and HAI-1 was not detected, or found at negligible levels, in stromal-derived ovarian tumors and various human sarcomas. In contrast, matriptase and HAI-1 were detected in all of the human carcinoma specimens in this study.

The detection of matriptase mRNA by in situ hybridyzation in primary human breast tumors revealed that the matriptase/HAI-1 system is synthesized by epithelial cells and epithelium-derived cancer cells in vivo. The lack or negligible amount of matriptase in stromal-derived ovarian tumors and primary human sarcomas, including osteosarcoma, liposarcoma, leiomyosarcoma, malignant fibrous histiocytoma and dermatofibrosarcoma protuberans, further confirm that epithelial cells, rather than mesenchymal cells, are the major source of matriptase in vivo. These results are consistent with our earlier observation that matriptase and HAI-1 are produced in vitro by breast cancer cells and milk-derived, immortalized luminal epithelial cells of the mammary gland, but not by cultured foreskin fibroblasts nor the fibrosarcoma cell line HT1080²⁶.

When assayed by western blotting of tumor cell lysates and normal tissue lysates, matriptase protein is present in tumor tissue and in normal breast tissue. Direct comparison of matriptase protein levels between tumor and surrounding normal tissue by western analysis, however, is of limited value due to cellularity issues, since tumors tend to contain primarily epithelial cells whereas normal breast tissue is composed primarily of stromal tissue with a smaller epithelial component. When examined by immunohistochemistry, there is no obvious difference in the expression of matriptase between tumor cells and normal breast epithelial cells (FIG. 6). This is similar to the observation for cultured immortalized/non-tumorigenic breast epithelial cells and tumorigenic breast cancer cell lines for which no obvious overall difference exists in the level of expression of matriptase mRNA and protein.

These results suggest that if the catalytic activity of the serine protease matriptase is important for the growth and/or invasion of breast cancer cells in human breast tumors, then the increased activity of the protease in breast cancer is likely due to mechanisms other than a simple increase in matriptase protein or mRNA. An increase in matriptase activity could be manifested in multiple ways, for example, by an increase in the matriptase:inhibitor ratio within a tumor, tipping the balance in favor of the protease relative to the inhibitor, or by an increase in the activation of matriptase on the cell surface by proteolytic cleavage. Like many other proteases, matriptase requires proteolytic cleavage from a one-chain latent form to a two-chain active form^{a}, an event that is not measured by the immunohistochemistry or in situ hybridization assays presented in this paper.

A significant imbalance of matriptase and HAI-1 expression was observed in some of the tumor specimens analyzed by western blot in this study. We have determined the ratio of matriptase to HAI-1 expression by comparing their intensity on western blot after normalizing the signals with that of the control sample from T-47D cells (lane 1 in FIGS. 2, 3, and 4) (Data not shown). These ratios fluctuate among the breast and gynecological tumors. For example, two infiltrating carcinomas (FIG. 2 lanes 4 and 6) had a relatively low ratio, whereas other samples, all invasive carcinomas, had a relatively higher ratio. Among the gynecological tumors, a high ratio was observed in some specimens (FIGS. 3A and B, lanes 10 and 11, and FIGS. 3C and D, lanes 3 and 7), but not in others. The matriptase to HAI-1 ratio among the colon tumors analyzed showed a more consistent value, with one exception (FIG. 4, lane 9). Taken together, these results suggest that the ratio of Matriptase to HAI-1 varies among tumors of breast and gynecological origin, and warrants further study to determine if a trend in the matriptase to HAI-1 ratio, assessed in a much larger set of tumors, correlates with pathological grade or stage of the tumors, or with clinical measures of outcome such as disease-free and overall survival or response to chemotherapy. Such studies are currently in progress.

In summary, we have characterized the expression of matriptase and HAI-1 both in cultured human breast cancer cells and in primary human breast carcinomas and other primary human cancers. We have found that matriptase and HAI-1 are expressed concomitantly by both ER− and ER+ breast cancer cell lines. The expression of these proteins correlates with the expression of epithelial markers and not with markers of a mesenchymal phenotype in cultured breast cancer cells. In primary breast cancers and in normal breast epithelial tissue, matriptase and HAI-1 are expressed by the epithelial component of the tissue, and not by stromal elements such as fibroblasts and adipocytes. The expression pattern of matriptase and HAI-1 in primary breast cancers suggests that this protease system is an epithelial-derived system that may activate stromal-derived proteases such as uPA, and growth/motility factors such as HGF/SF, on the surface of breast cancer cells, enhancing their growth and/or invasive properties. Therefore, matriptase may represent an important link in our understanding of how stromally-derived proteases and growth/motility factors may be activated on the surface of normal breast epithelial cells or breast cancer cells. Within a breast tumor, such activity may contribute to the tumorigenic and metastatic properties of breast cancer cells.

II. Regulation of the Activity of Matriptase on Epithelial Cell Surfaces by a Blood-Derived Factor

Materials and Methods

Cell lines and culture conditions: 184 A1N4 cells (A1N4, provided by Dr. M. R. Stampfer, U.C. Berkeley) [46] and MCF-10A cells (Michigan Cancer Foundation, Detroit, Mich.) are immortalized, non-tumorigenic, human mammary epithelial cells. A1N4 cells were maintained in Iscove's Modified Dulbecco's Medium (IMEM) (Gibco BRL, Rockville, Md.), supplemented with 0.5% fetal bovine serum (Gibco BRL), 0.5 g/ml hydrocortisone (Sigma, St Louis, Mo.), 5 g/ml insulin (Biofluids, Rockville, Md.) and 10ng/ml epidermal growth factor (EGF) (Collaborative Biomedical Research, Waltham, Mass.). MCF-10A cells were maintained in 50; 50% IMEM:HAM F12 (GIBCO BRL) supplemented with 5% horse serum, 0.5 μg/ml hydrocortisone, 5 g/ml insuline and 10 ng/ml EGF.

Purification of matriptase from human milk and from the conditioned medium of T-47D breast cancer cell: To purify complexed matriptase (two-chain form), human milk was fractionated by CM-Sepharose chromatography, and the 95-kDa matriptase complex fractions were then loaded onto an anti-matriptase mAb 21-9-Sepharose immunoaffinity column, as described previously [40]. Bound proteins were eluted by 0.1 M glycine buffer, pH 2.4, and stored in this low pH condition. To purify uncomplexed matriptase (one-chain form) from serum-free T-47D cell-conditioned medium, the complexed matriptase and HAI-1 were first depleted by passing the condition medium through an anti-HAI-1 mAb M58-Sepharose column. The unbound fraction (flow through) was further loaded onto a 21-9-Sepharose column, and bound proteins were eluted by 0.1 M glycine buffer pH 2.4, as described previously [39]. The eluted proteins were stored in low pH to prevent their degradation. To investigate the expression of matriptase in human urine, fresh urine was concentrated by 50-fold, and then examined by western blot using mAb M32.

Diagonal gel electrophoresis: Matriptase samples purified from T-47D cells and human milk were subjected to non-reduced/reduced diagonal gel electrophoresis. In the first dimension, matriptase preparations were boiled in SDS sample buffer in the absence of reducing agents and resolved by SDS gel electrophoresis. A gel strip was sliced out, boiled in SDS sample buffer in the presence of reducing agents, and electrophoresed on a second SDS polyacrylamide gel.

Amino acid sequence analysis of the 45- and 25-kDa fragments of matriptase: Milk-derived 95-kDa matriptase complexes were purified using a combination of CM-Sepharose chromatography and anti-matriptase mAb 21-9-Sepharose immunoaffinity chromatography, as described above. Both 45- and 25-kDa fragments of matriptase were resolved by non-reduced/reduced diagonal gel electrophoresis, as described above, and then transferred to polyvinylidene fluoride (PVDF) membranes. The amino-terminal sequences of these two fragments were determined as described previously [47] in the Howard Hughes Medical Institute Biopolymer Laboratory & W. M. Keck Foundation Biotechnology Resource Laboratory at Yale University.

Proteolytic activity assay: The proteolytic activity of matriptase was assayed at 25° C. by incubating matriptase in 200 l of 20 mM Tris buffer, pH 8.5, containing 0.1 mM of N-tert-butoxy-carbonyl (N-t-BOC)-Gln-Ala-Arg-7-amino-4-methylcoumarin (Sigma, St. Louis), as a substrate. The rate of cleavage was determined with a fluorescence spectrophotometer (Hitachi, F-4500).

Production of mAbs which are directed against matriptase: A panel of hybridoma lines, secreting mAbs directed against matriptase, were generated in our previous study [40]. These hybridoma lines were initially selected for mAbs that are able to recognize the 95-kDa matriptase/HAI-1 complex under non-boiled conditions and that additionally recognize the 70-kDa matriptase after boiling.

Immunoblotting analysis: Immunobloting was conducted as previously described [39]. Proteins were separated by 10% SDS-PAGE, transferred overnight to nitrocellulose sheets (Schleicher & Schuell, Keene, N.H.) or polyvinylidene fluoride (PVDF) and subsequently probed with mAb, as indicated. Immuno-reactive polypeptides were visualized using peroxidase-labeled anti-rat immunoglobulin and the ECL detection system (NEN, Boston, Mass.).

Induction of matriptase activation in A1N4 mammary epithelial cells: To serum starve 184 A1N4 cells, the cells were plated at 50-60% confluence, and maintained for 48-72 hrs in medium containing 0.5% serum. The activation of matriptase was then induced by incubating the cells with Iscove's Modified Dulbecco's Medium (IMEM), containing serum, as indicated in the Figure legend. Cells were then scrapped in PBS, and pelleted by centrifigation with a 5 min spin (1500×g). The cell pellets were lysed on ice for 20 min in lysis buffer (1% TritonX-100 in phosphate buffered saline). Cellular debris were removed by centrifugation for 10 min at 14,000×g. Equal protein amounts, as determined by the BCA protein micro assay (Pierce), were resolved under non-reducing, non-boiled conditions, by sodium dodecyl sulfate-polyacrylamide gel electrophoresis (SDS-PAGE), and electroblotted onto PVDF membranes (Millipore).

Immunofluorescence: Cells were fixed with 2% paraformaldehyde for 10 min. The two-chain form matriptase was detected with mAb M69 and total matriptase was detected with mAb M32 for 1 hr at room temperature. This was followed by an 1 hr incubation with 1:200 dilution of FITC-conjugated goat anti-mouse (Jackson Immunoresearch, PA). Cells were viewed on a Zeiss microscope, and photographed with Kodak film.

Results

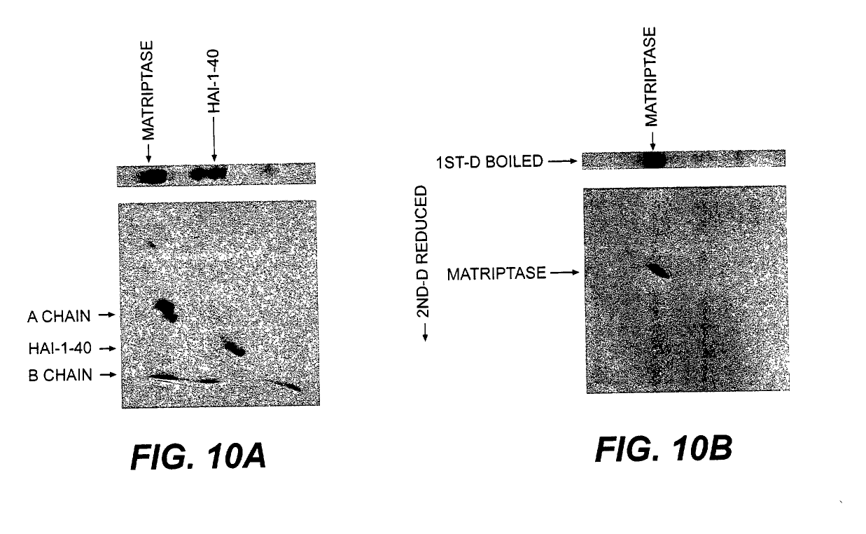

Matriptase is expressed both in single chain and two chain forms—We have previously shown that in human milk, the majority of matriptase is associated with its cognate Kunitz-type inhibitor, HAI-1, forming a 95-kDa complex [40]. In contrast, in T-47D human breast cancer cells, matriptase was mainly present as a 70-kDa, uncomplexed form, although the matriptase/HAI-1 complex was also observed [39]. Since in T-47D breast cancer cells, HAI-1 was primarily detected in its uncomplexed form, this observation suggested that the majority of the matriptase present in the condition media for this cell type lacks binding affinity to HAI-1. Most serine protease inhibitors, with a few exceptions, first require the cleavage of the target protease at a canonical activation motif, resulting in the formation of the substrate-binding pocket. Only then can these serine proteases associate with the inhibitors that block their activity. Therefore, the lack of interaction between matriptase and HAI-1 observed in T-47D cells, could be explained by the fact that the majority of matriptase is present in the single-chain, zymogen form. In contrast, the complexed matriptase, isolated from human milk, is likely to be in its activated, two-chain form. To test this hypothesis, we isolated complexed matriptase from human milk, and uncomplexed protease from the conditioned medium of T-47D cells. Both matriptase preparations were subjected to non-reduced/reduced diagonal gel electrophoresis (FIG. 10). In this electrophoresis assay, proteins that contain multiple disulfide-linked components will dissociate into their subunits, whereas a single-chain will remain as a single entity. As seen in FIG. 10A, the 95-kDa matriptase-complex derived from milk was converted to the 70-kDa matriptase and the 40-kDa fragment doublet of HAI-1 under boiled but non-reduced conditions (FIG. 10 panel A, 1^{st} D). When these dissociated proteins were electrophoresed under reduced conditions, the 70-kDa matriptase separated into two groups of polypeptides with apparent sizes of 45-kDa (A chain) and 25-kDa (B chain) (FIG. 10 panel A, 2^{nd} D). In contrast, the uncomplexed matriptase purified from T-47D cells remained as a single chain, with an apparent size of 80-kDa (FIG. 10B). The increase in its size probably results from the reduced rate of migration after treatment with reducing agents. These results suggest that matriptase present in the 95 kDa matriptase/HAI-1 complex consists of two-chains, whereas the uncomplexed matriptase from T47D conditioned medium is a single-chain protein.

To determine the position of the cleavage site for the generation of the two-chain form of matriptase, the 45- and 25-kDa chains were each subjected to N-terminal amino acid sequence analyses. The 25-kDa B chain contains the VVGGTDADEGEWP amino acid sequence at its amino terminus. This sequence begins with the likely cleavage site within the activation motif of matriptase. When the 45-kDa A chain (including two major spots and one minor spot present in the diagonal gel) was sequenced, two overlapping sequences (SFVVTSVVAFPTDSKTVQRT; TVQRTQDNSCSFGLHARGVE) were obtained, and both matched sequences close to the amino terminus of matriptase. These two different amino-terminal sequences may be derived from the two major spots of matriptase A chain, and suggest that the different migration rates of the two components result from different amino termini.

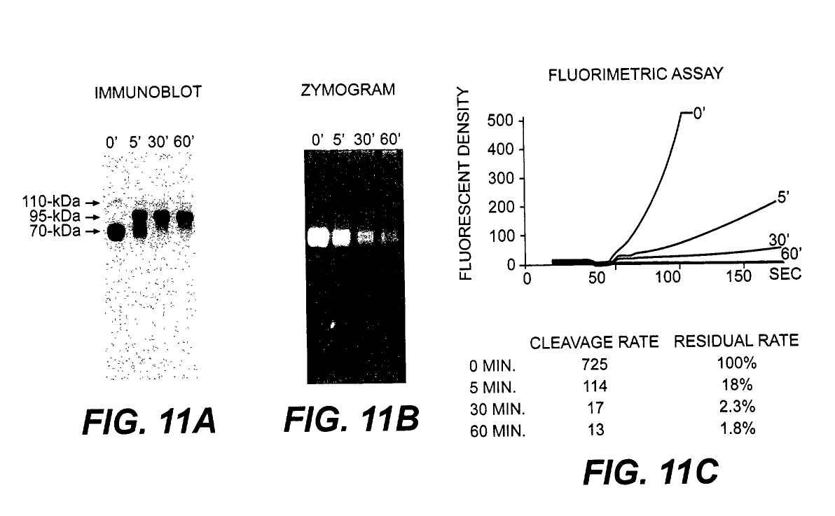

HAI-1 binds and inhibits matriptase—We have shown above that HAI-1 can form stable complexes with the two-chain form of matriptase (FIGS. 10 and 11). To demonstrate that HAI-1 inhibits the activity of matriptase, the matriptase/HAI-1 complexes were purified from human milk, as described in our previous study [40], and maintained in pH 2.4 to prevent the association of HAI-1 with matriptase. As the pH of the solution was raised to pH 8.0 and incubated at 37° C., the formation of 95-kDa matriptase/HAI-1 complex rapidly occurred (FIG. 11A). Binding of HAI-1 to matriptase was reflected by the shift of matriptase from the 70-kDa uncomplexed form, to the 95-kDa matriptase/HAI-1 complex. Uncomplexed matriptase became undetectable by immunoblot after 30 min. of incubation (FIG. 11A). While strong gelatinolytic activity was observed for the 70-kDa two-chain uncomplexed matriptase in a gelatin zymogram (FIG. 11B), only trace amounts of gelatinolytic activity could be detected for the 95-kDa matriptase/HAI-1 complex. This low level of proteolytic activity observed for the 95 kDa complex could result from excessive levels of substrate (1 mg/ml of gelatin) present in the zymogram, since the Kunitz-type serine inhibitors are known to bind to and to inhibit serine proteases in a reversible, competitive mode. Furthermore, the rate of cleavage of a synthetic, fluorescent substrate by matriptase was drastically decreased, following binding of HAI-1 to matriptase (FIG. 11C). These results provide direct evidence that the two-chain form of matriptase displays proteolytic activity and that binding of HAI-1 results in its catalytic inhibition, which is acid sensitive and reversible.

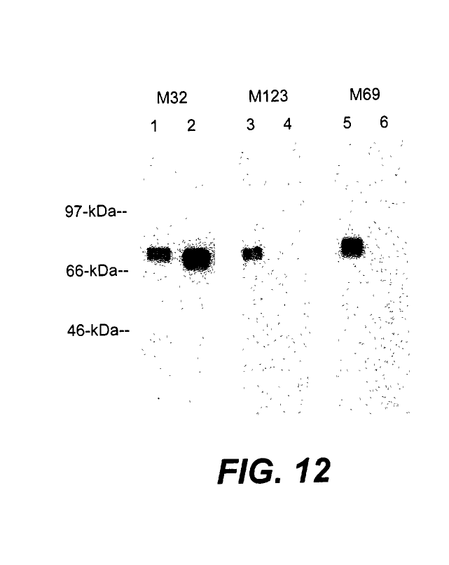

Production of mAbs which are specifically directed against the active, two-chain matriptase—In order to investigate the mechanism of activation of matriptase, we have generated monoclonal antibodies (mAb) which can distinguish the activated, two-chain form of matriptase from the single-chain zymogen of matriptase. Proteolytic cleavage of a single, specific peptide bond in the canonical serine protease activation motif, which transforms catalytically inactive serine proteases into their active forms, results in discrete, highly localized conformational changes [48]. Therefore, mAbs directed against these activation-induced conformational changes of matriptase can differentiate the active form from the latent form of the enzyme. We had previously generated more than 80 hybridoma lines, using the 95-kDa matriptase/HAI-1 complex as an immunogen [40]. In the current study, we have further selected two anti-matriptase mAbs, M69 and M123, which are both able to specifically distinguish the two-chain form of matriptase from the single-chain form of the protease. As shown in FIG. 12, mAb M32 detects both purified two-chain and single-chain matriptase forms (lanes 1 and 2). In contrast, mAbs M123 (FIG. 12 lane 3) and M69 (FIG. 12 lane 5) only recognize the two-chain form of the protease; they did not recognize the single-chain form of matriptase (FIG. 12 lanes 4 and 6). In addition to western blotting analysis, these mAbs are powerful tools to specifically detect the activated form of matriptase in intact cells and tissues.

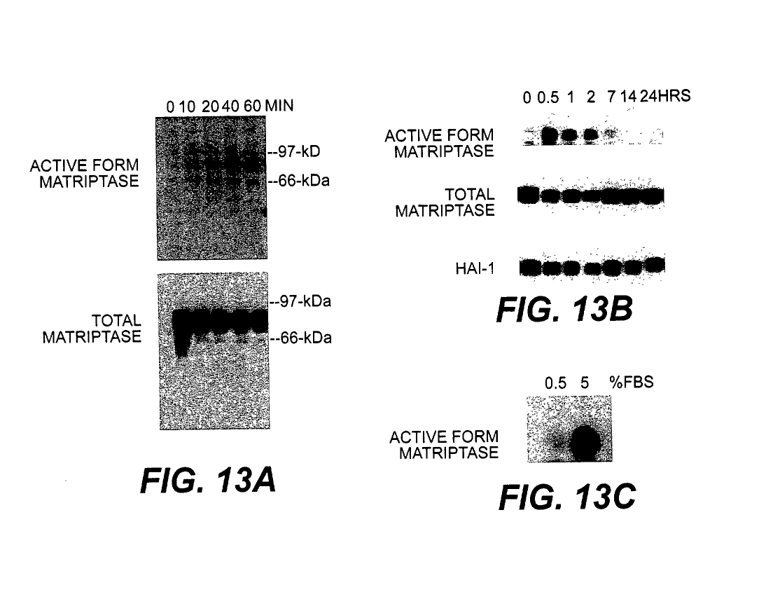

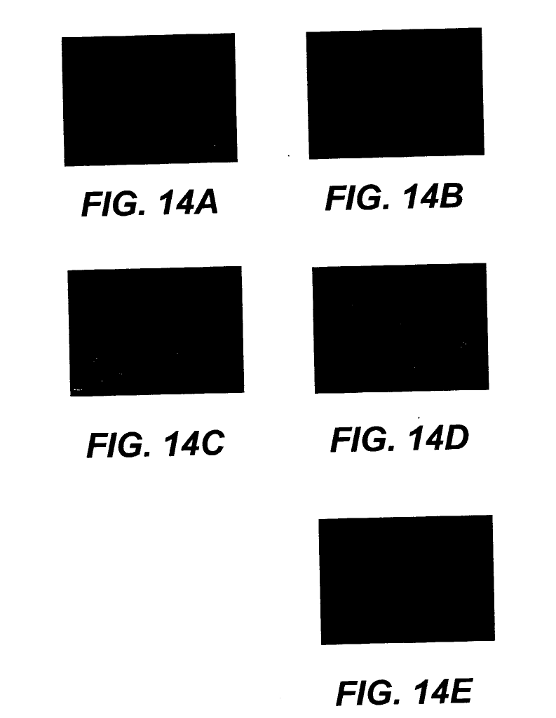

Transient activation of matriptase by sera in human mammary epithelial cells—We used these two-chain-specific anti-matriptase mAbs, together with anti-total matriptase mAbs, to explore the activation of matriptase. Only negligible levels of the two-chain form of matriptase were detected by western blotting, when non-tumorigenic 184 A1N4 human mammary epithelial cells were maintained for 2 days under low serum conditions. This observation suggests that most of the matriptase is expressed as the single-chain zymogen form in serum-starved A1N4 cells (FIGS. 13A and B, time 0). Exposing the cells to fresh fetal bovine serum results in a sharp increase in the level of the two-chain form of matriptase. This increase in the level of the two-chain form occurred within 10 min of serum stimulation (FIG. 13 panel A and B), and was maintained for up to 7 hrs, at which time the level of the two-chain form strongly decreased (FIG. 13B). The concentration of serum, rather than the availability of matriptase, was the limiting factor for the activation of matriptase, since the duration of the activation of matriptase depends on the amount of serum added to the cells. Active matriptase was still detectable 16 hr after serum stimulation when cells were treated with 5%, instead of 0.5% of fetal bovine serum (FIG. 13C). In addition to the 70-kDa two-chain form of matriptase, we also detected in A1N4 cells the two-chain form of matriptase complexed with HAI-1 (data not shown). Immunofluorescence staining confirmed that exposure to serum induces the formation of two-chain form matriptase, which is localized at the surface of A1N4 cells (FIG. 14). Serum also induced the activation of matriptase in an independently derived immortalized human mammary epithelial cell line, MCF-10A (data not shown). Interestingly, the T47D breast cancer cells did not increase their level of two-chain form of matriptase in response to serum (data not shown). These results suggest that serum contains a factor which can induce activation of matriptase in human mammary epithelial cells. This factor could be consumed or inactivated by the cells, resulting in the transient activation of matriptase.

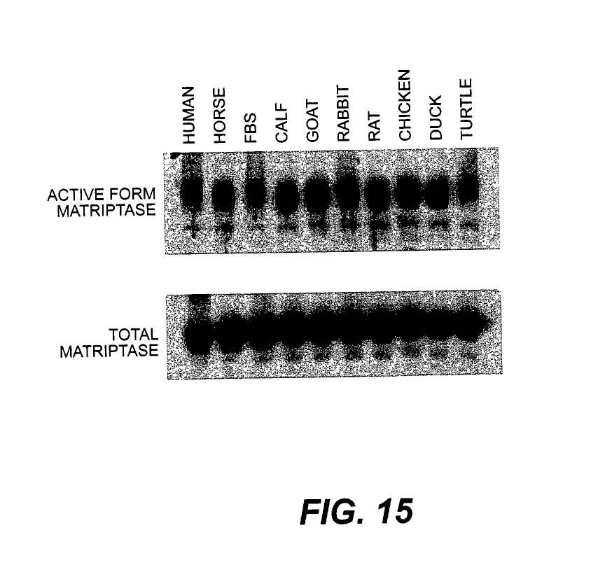

Activation of matriptase can be induced by sera from various animals—In addition to fetal bovine serum, sera from human, horse mouse, rat, rabbit, duck, chicken, goat, calf, and even turtle were all able to induce matriptase activation in A1N4 (FIG. 15). These results suggest that a blood-based mechanism for activation of matriptase could be evolutionarily conserved.

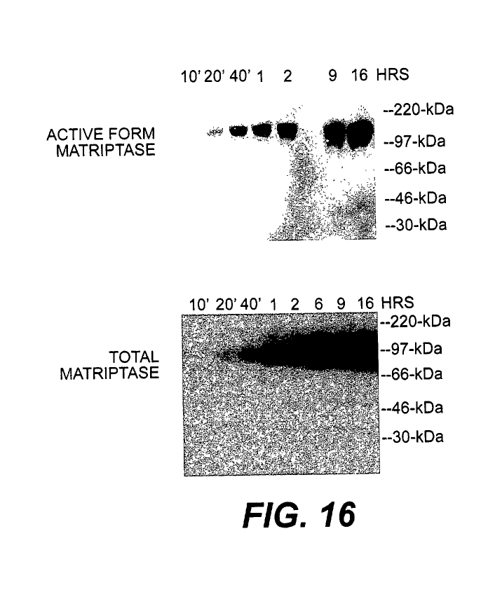

Activation of matriptase is accompanied by the release of matriptase and its inhibitor from the surface of cells—Further examination of the expression of total matriptase and its inhibitor was carried out as FBS was added to the cells: results showed an inverse correlation between the level of the two-chain form and total matriptase. As the level of the two-chain matriptase was increased in A1N4, the level of total matriptase was reduced (FIG. 13B). Interestingly, as the level of active matriptase diminished, the amount of total matriptase increased (FIG. 13B). Expression of HAI-1 in A1N4, after addition of serum, paralleled that of total matriptase (FIG. 13B). Since matriptase can be detected in cell condition medium, the decrease in the levels of total matriptase, accompanying its activation, could be explained by its release from the surface of cells (ectodomain shedding). Indeed, total matriptase and its two-chain form accumulated in the cell-conditioned medium following stimulation with serum (FIG. 16). These results suggest that in A1N4 cells, serum induces ectodomain shedding, both of matriptase and of HAI-1, in addition to the induction of the activation of matriptase.

Discussion

Matriptase is a potent, trypsin-like protease, which serves as an activator for other proteases, growth factors, and receptors on the surfaces of epithelial cells. Its activity must, therefore, be tightly regulated. First, we have shown that HAI-1 binds and inhibits the proteolytic activity of the two-chain active form of matriptase. We hypothesized that the proteolytic activation of matriptase would be an irreversible process, similar to most other proteases. Thus, its cognate inhibitor, HAI-1, could play a major role in preventing unwanted, prolonged proteolysis, once matriptase is activated. The current study describes the discovery of a serum component(s), which can induce the activation of matriptase on the surface of mammary epithelial cells. The serum-induced activation of matriptase is transient, suggesting that this blood-derived inducer can be consumed or inactivated by epithelial cells. Therefore, the availability of this inducer, such as during physiologic or pathologic states of tissue remodeling, could provide an initial stimulus for the regulation of the activation of matriptase.

Once activated, binding of matriptase to HAI-1 and its shedding from the surface of cells could be essential steps for its inactivation and clearance. Indeed, the 95-kDa matriptase/HAI-1 complex represents the major form of matriptase present in milk [40] and in urine (data not shown), and this complex is likely to be a final product following matriptase activation, shedding, and/or inhibition. Because the shedding of matriptase follows its activation, this ectodomain shedding may serve along with HAI-1 inactivation to prevent prolonged retention of proteolytic activity on the surface of cells. Although we used mammary epithelial cells to characterize these regulatory mechanisms, they are also most likely involved in the modulation of the activity of matriptase in other epithelial cell types, since the matriptase/HAI system is expressed by various epithelia [41-43].

This blood-based regulatory mechanism for the activation of epithelial proteolytic systems may play an important role in the regulation, maintenance, and repair of the epithelium. Since the epithelium is separated from its underlying connective tissue by the basement membrane, blood vessels do not normally penetrate the epithelium. To reach the epithelium, serum proteins and nutrients must pass through the capillary walls into the surrounding connective tissue and through the basement membrane. Therefore, the activation of matriptase may be regulated by the rate of influx of blood. In some circumstances, such as the lactating mammary gland, proteins from the plasma are transported by transcytosis into the milk. Thus, this large influx of blood through the lactating mammary gland may be expected to promote the activation of matriptase on mammary epithelial cells. Results from our previous studies, together with those presented here [40], indicate that the predominant form of matriptase in human milk is the two-chain form, tightly bound with its cognate, Kunitz inhibitor. The discovery of a blood-based mechanism for the activation of matriptase provides a clue for explaining the increase in the activation of matriptase, observed in the lactating mammary gland. Large-scale exposure of epithelial cells to blood components also occurs in kidney, where the expression of matriptase has been reported [42, 43]. Thus, serum-dependent activation, HAI-1 inhibition, and shedding of matriptase could also occur in kidney. This hypothesis is supported by detection of 95-kDa matriptase/HAI-1 complex in human urine (data not shown). In addition, matriptase may be important for the process of wound healing, which involves extensive extracellular matrix-degradation and cell migration. Matriptase and HAI-1 are expressed in epidermal cells, as examined by immunohistochemistry (data not shown). The yet uncharacterized serum component(s) we have described here could induce the activation of matriptase, which in turn could activate uPA and HGF in the early stages of wound healing. Both uPA and HGF have been implicated in ECM degradation and cell motility, two key events in tissue remodeling and wound healing. Active matriptase then may be quickly inhibited through the binding of HAI-1. As the basement membrane is reformed, the activation of matriptase may then be reduced, since the basement membrane functions as a barrier for blood influx into the epithelium. In contrast to non-transformed mammary epithelial cells (A1N4, MCF-10A), breast cancer cell lines (such as T47D) are insensitive to serum, with respect to the activation of matriptase; however, they constitutively maintain low levels of the active form of matriptase, even under serum-depleted conditions (data not shown) [39]. These observations suggest that the blood-derived factor we have described here might reflect a physiological process. Furthermore, as epithelial cells acquire malignant transformation, they may lose this mechanism of transient regulation of the activation of matriptase, but gain constitutive expression of the proteolytic active form of matriptase on their surface (manuscript in preparation).

The mechanism by which serum induces the activation of matriptase still needs to be elucidated. Matriptase contains a canonical serine protease activation motif and a proteolyitc cleavage site at Arg-Val, both of which are likely to be required for its activation. When expressed in E. coli, the serine protease domain of matriptase can be autoactivated [42]. Although autolytic activation is not the usual case for most serine proteases, it does occur in some instances, such as the complement C1r protease [49, 50]. The C1r protease contains a CUB-EGF module, which is thought to be important for the protein-protein interaction and autolytic activation of C1r. Considering that matriptase contains CUB domains, and that autolytic activation of the serine protease domain of matriptase can occur in vitro, autolytic activation is thus a potential mechanism for the activation of matriptase. If this is the case, the serum component(s) may act as a C1q-like molecule to transduce the activatory signal, stimulating the autoactivation of matriptase. Alternatively, the serum factor could trigger a proteolytic cascade on the surface of epithelial cells, resulting in the proteolytic activation of matriptase, or it may be a protease that directly activates matriptase.

Activated matriptase is removed from the cell surface by ectodomain shedding, providing an additional means to regulate the amount of protease and the degree of proteolytic activity on the surfaces of epithelial cells. The amino terminal sequences of the two A chains of matriptase were determined to be SFVVTSVVAFPTDSKTVQRT and TVQRTQDNSCSFGLHARGVE; thus, the cleavage sites are located between 266Lys-Ser 267 and 281Lys-Thr282 (see updated GenBank™/EBI Data Bank with accession number AF118224). These results suggest that a still unidentified protease, with cleavage preference between Lys and amino acid residues containing aliphatic hydroxyl side chains, may be responsible for the shedding of matriptase.

In conclusion, we have described a novel, blood-derived, evolutionarily conserved mechanism for the activation and regulation of an epithelial, membrane-bound, serine protease. The activated matriptase can, in turn, activate pro-uPA, a major stromal ECM-degrading protease system, HGF/SF, a prominent stromal-derived epithelial motility factor in the close vicinity of the cell surfaces, and PAR2, a cell surface receptor. The presence of the Kunitz-type inhibitor, HAI-I, prevents prolonged proteolytic activity matriptase. Future studies will be required to establish a more direct relationship between matriptase activation, ECM degradation, and epithelial motility. Matriptase may be activated in vivo by the contact of blood with epithelial surfaces: downstream effectors of matriptase may serve a role in communication between epithelial and stromal cells.

III. Induction of Protease Activation by Bioactive Lipids on the Surface of Epithelial Cells

Bioactive lipids such as lysophosphatidic acid (LPA) and shingosine 1-phosphate (S1P) have pleiotropic cellular effects, including proliferation, survival, cytoskeletal rearrangement, and migration. We are now describing a novel biological function of LPA and S1P: the activation of epithelial cell surface protease, matriptase. The lipid fraction of lipoproteins, LPA, and S1P specifically induce the rapid activation of matriptase on the surface of mammary epithelial cells (possibly keratinocytes, smooth muscle cells and ovarian cells). These results provides a critical missing link in the proposed role of LPA and S1P in normal tissue remodeling and pathology.

Matriptase (also known as membrane type serine protease 1, MT-SP1) is a membrane protease expressed on the surface of a variety of epithelial cells, where it can functions as an activator of stromal-derived effectors involved in tissue remodeling. Matriptase has been demonstrated to activate the urokinase-type plasminogen activator, the hepatocyte growth factor (HGF)/scattering factor (SF), and the protease activated receptor 2 (PAR 2), which are implicated in tissue remodeling, induction of cell motility and calcium influx respectively. Therefore, matriptase serves as an activator of important effector molecules involved in a variety physiological and pathological processes, such as tissue remodeling, inflammation and cancer invasion and metastasis. However, the regulation of the activation of matriptase is not yet understood.

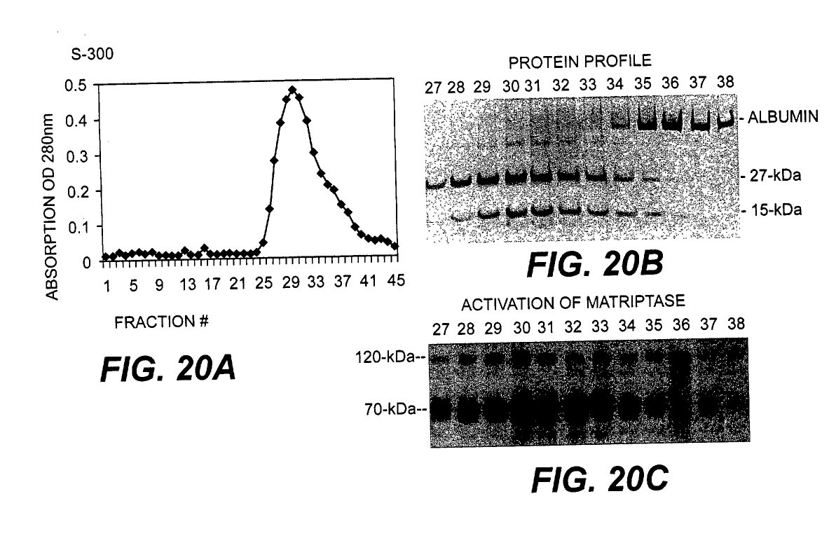

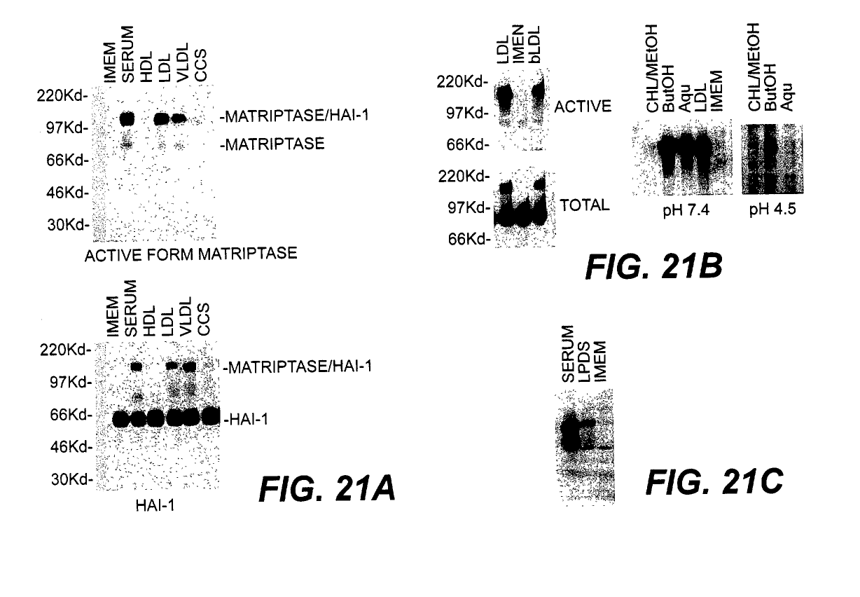

Matriptase is synthesized as a single-chain zymogen and presented on the surfaces of cells were it is activated . Activation of matriptase results in conformational changes, creating new immunological epitopes. Using an antibody that specifically recognizes the active two-chain form of matriptase, we have recently demonstrated that serum induces the activation of matriptase. To identify the factor(s) present in serum responsible for the induction of matriptase, we fractionated human serum by DEAE chromatography, and removed albumin by Cibarco Blue Dye-Agarose. Further purification and determination of the size of the serum factor was carried out by S-300 gel filtration column (FIG. 20). The activity was associated with a protein complex with an approximate size of 160-kDa. This complex contains several subunits with sizes of 27 and 15 KDa under non reduced conditions. A similar active 160-kDa complex was also purified using different chromatography, such as zinc chelating column, hydroxyapitide chromatography. Immunoaffinity chromatography, using mAb which is directed against this complex, was used to confirm that this complex induced the activation of matriptase. The MALDI-MS analysis identified the 27-kDa subunit of the 160-kDa complex as apolipoprotein A, indicating that the purified factor is a lipoprotein. The ability of lipoproteins to induce the activation of matriptase was confirmed by treating human mammary epithelial cells with commercial lipoproteins (FIG. 21). Both LDL and VLDL induced the activation of matriptase to the same extend as serum (FIG. 21A). Furthermore, charcoal stripped serum, which is depleted of lipids, failed to induce matriptase activation. Activated matriptase was detected in whole cell lysates as both a complex with its inhibitor HAI-1 (120-kDa) and as a non-complexed form (70-kDa) (FIG. 21). The activation potential of LDL was not destoyed by boiling (FIG. 21B), or protein acetylation, which inactivates apolipoproteins function, suggesting that the factor is a lipid component of lipoproteins. Further extraction of LDL and serum with organic solvents suggested that a phospholipid was inducing the activation of matriptase (FIG. 21B). In addition, lipoprotein deficient serum still contained some activity. (FIG. 21C)

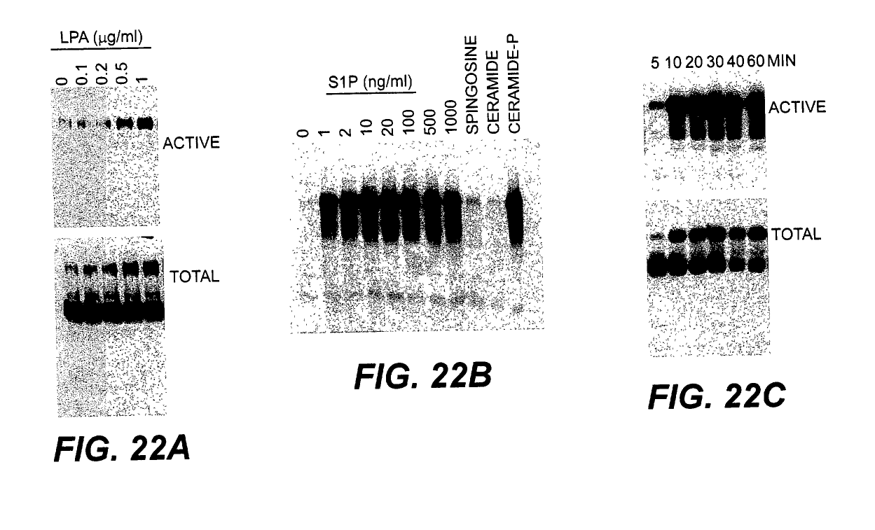

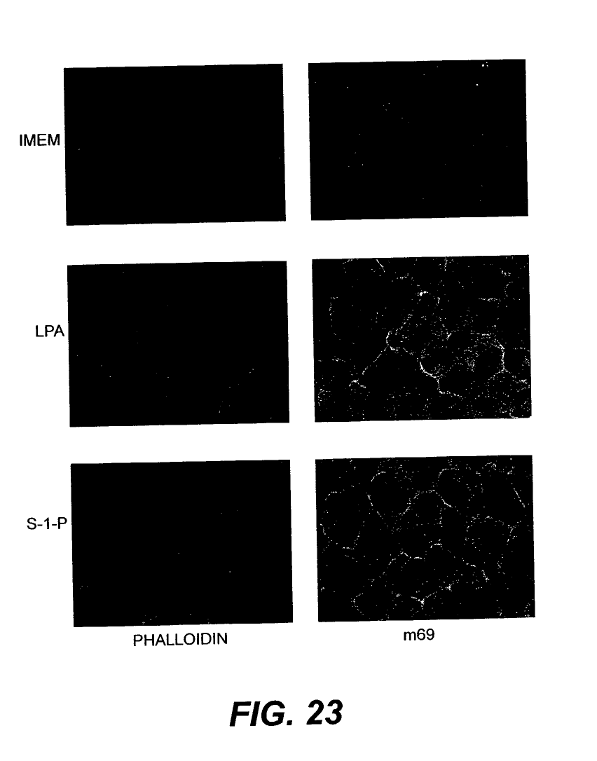

Previous studies have shown that lipoproteins are carriers of phospholipides including LPA and S1P. We had observed that our purified serum-derived fraction, as well as commercial LDL and VLDL induced rapid cytoskeletal rearrangement of A1N4 cells, a biological effect wildly reported to be induced in fibroblasts by LPA and S1P. We, therefore, tested the ability of LPA and S1P to induced the activation of matriptase in intact A1N4 cells. Both LPA and S1P could mimic serum and LDL induced activation of matriptase (FIGS. 22A and B). However, S1P was 100 fold more potent than LPA in inducing the activation of matriptase. Maximal activation was achieved at 1 ng/ml (2.6 nM) and 1 μg/ml (×M), S1P and LPA respectively. The activation of matriptase started within 5 min of S1P addition, reaching maximal activation within 10 min (FIG. 22C). The ability of LPA and SIP to induce matriptase activation was highly specific, since no phosphoglycerides related to LPA were active, nor were sphingosine and ceramide (table 2). Ceramide1-P also induced the activation of matriptase although at higher concentration than S1P. Immunofluorescence study indicated that the activated matriptase, following LPA and S1P treatment, was located at the surface of 41N4 epithelial cell (FIG. 23). Similarly to what have been described in fibroblasts, both S1P and LPA induced actin cytoskelaton reorganization (stress-fiber formation) in A1N4 human mammary epithelial cells (FIG. 23). Pretreatment of cells with cytochalasin D, which disrupt the actin cytoskeleton, failed to block the activation of matriptase by S1P. These results suggest that activation of matriptase did not depend on actin cytoskeleton rearrangement.

Although both LPA and S1P can mimic serum-induced activation of matriptase on the surface of A1N4 cells, S1P is likely to be the major blood-derived factor, particularly in our assay system. We have previously shown that 0.5% serum was sufficient to induce matriptase activation. Since the concentrations of LPA and S-1P in human serum have been estimated to be approximately 1 uM and 0.5 uM, respectively, 0.5% serum would contain 5 nM LPA and 2.5 nM S1P. Therefore, only S1P is present at high enough concentration in total serum to stimulate the activation of matriptase. However, both S1P and LPA are released by activated platelets. Since keratinocytes and vascular smooth muscle cells express matriptase, S1P and LPA-dependent activation of matriptase may play an important role in the process of wound healing. Furthermore, ovarian cancer acities contain elevated levels of LPA, therefore activation of matriptase on the surface of ovarian tumor cells may be implicated in ovarian metastasis. In addition, S1P may play an important role as a paracrine factor during inflamation process and particularly in cancer where it may involve mesemchemal-epithelial cell interations. Cytokines and growth factors induce the expression of sphingosine kinase, the enzyme reponsible for the formation of S1P. Increase levels of cellular S1P could result in its release, and act in a paracrine manner stimulating the activation of matriptase on adjacent epithelila cells.

Activation of most serine proteases requires proteolytic cleavage at serine protease canonical activation domain and mainly depends on other proteases. This mechanism is observed in some physiological processes, such as the digestive proteases, the mammalian blood coagulation cascade, and the complement system. Conceivably an external signal and a different mechanism other than activation by other proteases should be needed to induce and initiate the activation of the first protease in the cascade. The complement system provides an excellent example: the antibody/antigen complex as an external signal and autoactivation of C1r protease as an alternative mechanism for the activation of the first protease in complement system. For most other serine proteases and protease cascades, the external signal and the mechanism for activation of first protease are not defined. For the digestive proteases, although enterokinase is believed to be the first protease, the mechanism for its activation and the signal for induction of its activation are still unknown. Although whether matriptase is the first protease on the periphery of epithelial cells remains further investigation, S1P as an external factor may play as a systemic or local factor, which can regulate epithelial function by triggering activation of proteases, such as matriptase and subsequently activate their substrates, such as HGF and PARP2 on the periphery of epithelial cells

While the invention has been described in terms of preferred embodiments, the skilled artisan will appreciate that various modifications, substitutions, omissions and changes may be made without departing from the spirit thereof. Accordingly, it is intended that the scope of the present invention be limited solely by the scope of the claims provided below, including equivalents thereof

### References

1. Mignatti P, Rifkin D B: Biology and biochemistry of proteinases in tumor invasion. Physiol Rev 1993, 73:161-195

2. Behrendt N, Ronne E, Dano K: The structure and function of the urokinase receptor, a membrane protein governing plasminogen activation on the cell surface. Biol Chem Hoppe Seyler 1995, 376:269-279

3. Conese M, Blasi F: Urokinase/urokinase receptor system: internalization/degradation of urokinase-serpin complexes: mechanism and regulation. Biol Chem Hoppe Seyler 1995, 376:143-155

4. Holst-Hansen C, Johannessen B, Hoyer-Hansen G, Romer J, Ellis V, Brünner N: Urokinase-type plasminogen activation in three human breast cancer cell lines correlates with their in vitro invasiveness. Clin Exp Metastasis 1996, 14:297-307

5. Hofmann R, Lehmer A, Buresch M, Hartung R, Ulm K: Clinical relevance of urokinase plasminogen activator, its receptor, and its inhibitor in patients with renal cell carcinoma. Cancer 1996, 78:487-492

6. Andreasen P A, Kjoller L, Christensen L, Duffy M J: The urokinase-type plasminogen activator system in cancer metastasis: a review. Int J Cancer 1997, 72:1-22

7. Sato, H, Takino, T, Okada, Y, Cao, J, Shinagawa, A, Yamamoto, E, Seiki, M: A matrix metalloproteinase expressed on the surface of invasive tumour cells. Nature 1994, 370:61-65

8. Stetler-Stevenson W G: Progelatinase A activation during tumor cell invasion. Invasion Metastasis 1994, 14:259-268

9. Basset P, Wolf C, Rouyer N, Bellocq J P, Rio M C, Chambon P: Stromelysin-3 in stromal tissue as a control factor in breast cancer behavior. Cancer 1994, 74:1045-1049

10. Wilson C L, Matrisian L M. Matrilysin: an epithelial matrix metalloproteinase with potentially novel functions: Int J Biochem Cell Biol 1996, 28:123-136

11. Okada, A, Bellocq J P, Rouyer, N, Chenard, M P, Rio, M C, Chambon, P, Basset, P: Membrane-type matrix metalloproteinase (MT-MMP) gene is expressed in stromal cells of human colon, breast, and head and neck carcinomas. Proc Natl Acad Sci U S A 1995, 92:2730-4

12. Nishida Y, Kohno K, Kawamata T, Morimitsu K, Kuwano M, Miyakawa I: Increased cathepsin L levels in serum in some patients with ovarian cancer: comparison with CA125 and CA72-4. Gynecol Oncol 1995 56:357-361

13. Nielsen B S, Sehested M, Timshel S, Pyke C, Dano K: Messenger rna for urokinase plasminogen activator is expressed in myofibroblasts adjacent to cancer cells in human breast cancer. Lab Invest 1996, 74:168-177

14. Pyke, C, Graem, N, Ralfkiaer, E, Ronne, E, Hoyer-Hansen, G, Brunner, N, Dano, K: Receptor for urokinase is present in tumor-associated macrophages in ductal breast carcinoma. Cancer Res 1993, 53:1911-1915

15. Polette, M, Gilbert, N, Stas, I, Nawrocki, B, Noel, A, Remacle, A, Stetler-Stevenson, W G, Birembaut, P, Foidart, M: Gelatinase A expression and localization in human breast cancers. an in situ hybridization study and immunohistochemical detection using confocal microscopy. Virchows Arch 1994, 424:641-645

16. Heppner K J, Matrisian L M, Jensen R A, Rodgers W H: Expression of most matrix metalloproteinase family members in breast cancer represents a tumor-induced host response. Am J Pathol 1996, 149:273-282

17. Dano, K, Romer, J, Nielsen, B S, Bjorn, S, Pyke, C, Rygaard, J, Lund, L R: Cancer invasion and tissue remodeling—cooperation of protease systems and cell types. APMIS 1999, 107:120-127

18. Jeffers M, Rong S, Woude G F: Hepatocyte growth factor/scatter factor-met signaling in tumorigenicity and invasion/metastasis. J Mol Med 1996, 74:505-513

19. Jiang W G, Hiscox S: Hepatocyte growth factor/scatter factor, a cytokine playing multiple and converse roles. Histol Histopathol 1997, 12:537-555

20. Matsumoto K, Nakamura T: Emerging multipotent aspects of hepatocyte growth factor. J Biochem (Tokyo) 1996, 119:591-600

21. Lee, S L, Dickson, R B, and Lin, C Y: Activation of hepatocyte growth factor and urokinase/plasminogen activator by matriptase, an epithelial membrane serine protease. J. Biol. Chem 2000, in press

22. Takeuchi T, Harris J L., Huang W, Yan K W, Coughlin S R, Craik C S: Cellular localization of membrane-type serine protease 1 and identification of protease activated receptor-2 and single-chain urokinase-type plasiminigen activator as substrates. J Biol Chem 2000, (in press)

23. Shi Y E, Torri J, Yieh L, Wellstein A, Lippman M E, Dickson R B: Identification and characterization of a novel matrix-degrading protease from hormone-dependent human breast cancer cells. Cancer Res 1993, 53:1409-1415

24. Lin C Y, Wang J K, Torri J, Dou L, Sang Q X A, Dickson R B: Characterization of a novel, membrane-bound, 80-kDa matrix-degrading protease from human breast cancer cells. J Biol Chem 1997, 272:9147-9152

25. Lin C Y, Anders J, Johnson M, Sang Q A, Dickson R B: Molecular cloning of cDNA for matriptase, a matrix-degrading serine protease with trypsin-like activity. J Biol Chem 1999, 274:18231-18236

26. Lin C Y, Anders J, Johnson M, Dickson R B: Purification and characterization of a complex containing matriptase and a kunitz-type serine protease inhibitor from human milk. J Biol Chem 1999, 274:18237-18242

27. Takeuchi T, Shuman M A, Craik C S: Reverse biochemistry: use of macromolecular protease inhibitors to dissect complex biological processes and identify a membrane-type serine protease in epithelial cancer and normal tissue. Proc Natl Acad Sci U S A 1999, 96:11054-11061

28. Kim, M G, Chen, C, Lyu, M S, Cho, E G, Park, D, Kozak, C, Schwartz, R H: Cloning and chromosomal mapping of a gene isolated from thymic stromal cells encoding a new mouse type II membrane serine protease, epithin, containing four LDL receptor modules and two CUB domains. Immunogenetics 1999, 49:420-428

29. Shimomura, T, Denda, K, Kitamura, A, Kawaguchi, T, Kito, M, Kondo, J, Kagaya, S, Qin, L, Takata, H, Miyazawa, K, Kitamura, N: Hepatocyte growth factor activator inhibitor, a novel kunitz-type serine protease inhibitor. J Biol Chem 1997, 272:6370-6376

30. Kataoka, H, Suganuma, T, Shimomura, T, Itoh, H, Kitamura, N, Nabeshima, K, Koono, M: Distribution of hepatocyte growth factor activator inhibitor type 1 (HAI-1) in human tissues. Cellular surface localization of HAI-1 in simple columnar epithelium and its modulated expression in injured and regenerative tissues. J Histochem Cytochem 1999, 47:673-682

31. Zhang Y, Cai X, Schlegelberger B, Zheng S: Assignment 1 of human putative tumor suppressor genes ST13 (alias SNC6) and ST14 (alias SNC19) to human chromosome bands 22q13 and 11q24—>q25 by in situ hybridization. Cytogenet Cell Genet 1998, 83:56-57

32. Mazzieri, R, Masiero, L, Zanetta, L, Monea, S, Onisto, M, Garbisa, S, Mignatti, P: Control of type IV collagenase activity by components of the urokinase-plasmin system: a regulatory mechanism with cell-bound reactants. EMBO J. 1997, 16:2319-2332.

33. Lamszus, K, Jin, L., Fuchs, A, Shi, E, Chowdhury, S, Yao, Y, Polverini, P J, Laterra, J, Goldberg, I D, Rosen, E M: Scatter factor stimulates tumor growth and tumor angiogenesis in human breast cancers in the mammary fat pads of nude mice. Lab Invest 1997, 76:339-353.

34. Sommers C L, Byers S W, Thompson E W, Torri J A, Gelmann E P: Differentiation state and invasiveness of human breast cancer cell lines. Breast Cancer Res Treat 1994, 31:325-335

35. Thompson, E W, Paik, S, Brunner, N, Sommers, C L, Zugmaier, G, Clarke, R, Shima, T B, Douglas-Jones, A G, Donahue, S, Lippman, M E, Dickson R B: Association of increased basement membrane invasiveness with absence of estrogen receptor and expression of vimentin in human breast cancer cell lines. J Cell Physiol 1992, 150:534-544

36. Pierceall W E, Woodard A S, Morrow J S, Rimm D, Fearon E R: Frequent alterations in E-cadherin and alpha- and beta-catenin expression in human breast cancer cell lines. Oncogene 1995, 11:1319-1326

[37] Lin, C. Y., Anders, J., Johnson, M., Sang, Q. A. & Dickson, R. B. (1999) Molecular cloning of cDNA for matriptase, a matrix-degrading serine protease with trypsin-like activity. Journal of Biological Chemistry, 274, 18231-18236.

[38] Shi, Y. E., Torri, J., Yieh, L., Wellstein, A., Lippman, M. E. & Dickson, R. B. (1993) Identification and characterization of a novel matrix-degrading protease from hormone-dependent human breast cancer cells. Cancer Research, 53, 1409-1415.

[39] Lin, C. Y., Wang, J. K., Torri, J., Dou, L., Sang, Q. X. A. & Dickson, R. B. (1997) Characterization of a novel, membrane-bound, 80-kDa matrix-degrading protease from human breast cancer cells. Journal of Biological Chemistry, 272, 9147-9152.

[40] Lin, C. Y., Anders, J., Johnson, M. & Dickson, R. B. (1999) Purification and characterization of a complex containing matriptase and a kunitz-type serine protease inhibitor from human milk. Journal of Biological Chemistry, 274, 18237-18242.

[41] Shimomura, T., Denda, K., Kitamura, A., Kawaguchi, T., Kito, M., Kondo, J., Kagaya, S., Qin, L., Takata, H., Miyazawa, K. & Kitamura, N. (1997) Hepatocyte growth factor activator inhibitor, a novel kunitz-type serine protease inhibitor. Journal of Biological Chemistry, 272, 6370-6376.

[42] Takeuchi, T., Shuman, M. A. & Craik, C. S. (1999) Reverse biochemistry: use of macromolecular protease inhibitors to dissect complex biological processes and identify a membrane-type serine protease in epithelial cancer and normal tissue. Proc. Natl. Acad. Sci. U.S.A., 96, 11054-11061.

[43] Kim, M. G., Chen, C., Lyu, M. S., Cho, E. G., Park, D., Kozak, C. & Schwartz, R. H. (1999) Cloning and chromosomal mapping of a gene isolated from thymic stromal cells encoding a new mouse type II membrane serine protease, epithin, containing four LDL receptor modules and two CUB domains. Immunogenetics, 49, 420-428.

[44] Takeuchi, T., Harris J. L., Huang W., Yan K. W., Coughlin, S. R. & Craik, C. S. (2000) Cellular localization of membrane-type serine protease 1 and identification of protease activated receptor-2 and single-chain urokinase-type plasiminigen activator as substrates. Journal of Biological Chemistry, 275, 26333-26342.

[45] Lee, S. L., Dickson, R. B. & Lin, C. Y. (2000) Activation of hepatocyte growth factor and urokinase/plasminogen activator by matriptase, an epithelial membrane serine protease. Journal of Biological Chemistry, 275, 36720-36725.

[46] Stampfer, M. R. & Bartley, J. C. (1985) Induction of transformation and continuous cell lines from normal human mammary epithelial cells after exposure to benzo[a]pyrene. Proc. Natl. Acad. Sci. U.S.A., 82, 2394-2398.

[47] Matsudaira, P. (1987) Sequence from picomole quantities of proteins electroblotted onto polyvinylidene difluoride membranes. Journal of Biological Chemistry, 262, 10035-10038.

[48] Blow, D. M. & Steitz, T. A. (1970) X-ray diffraction studies of enzymes. Annu. Rev. Biochem., 39, 63-100.

[49] Thielens, N. M., Bersch, B., Hernandez, J. F. & Arlaud, G. J. (1999) Structure and functions of the interaction domains of C1r and C1s: keystones of the architecture of the C1 complex. Immunopharmacology, 42, 3-13.

[50] Arlaud, G. J., Rossi, V., Thielens, N. M., Gaboriaud, C., Bersch, B. & Hernandez, J. F. (1998) Structural and functional studies on C1r and C1s: new insights into the mechanisms involved in C1 activity and assembly. Immunobiology, 199, 303-316.

What is claimed is:

1. An in vitro method of diagnosing the presence of a pre-malignant lesion, a malignancy, or other pathologic condition, in a subject, which is characterized by the presence of activated matriptase comprising the steps of:
(A) obtaining a biological sample from a subject that is to be tested for a pre-malignant lesion, a malignancy, or other pathologic condition;
(B) exposing the biological sample to a detectable agent which recognizes and binds to activated matriptase; and
(C) determining whether said detectable agent is bound to the biological sample.

2. The method of claim 1, wherein the detectable agent is an antibody.

3. The method of claim 2, wherein the antibody specifically binds to activated matriptase.

4. The method of claim 2, wherein the antibody is selected from M69 and M123.

5. The method of claim 2, wherein the antobody is radiolabeled.

6. The method of claim 5, wherein the labeled antibody is labeled with a radioisotope or a fluorescent label.

7. The method of claim 6, wherein the radioisotope is selected from the group consisting of: ⁶²Cu, ⁹⁹Te, ¹³¹I, ¹²³I, ¹¹¹In, ⁹⁰Y, ¹⁸⁸Re, and ¹⁸⁶Re.

8. The method of claim 2, wherein the method further comprises exposing the biological sample to one or more antibodies which do not specifically bind to active matriptase.

9. The method of claim 8, wherein at least one of the antibodies which do not specifically bind to active matriptase binds to the inactive form of matriptase.

10. The method of claim 8, further comprising determining the ratio between the antibody specifically bound to active matriptase and the total of bound antibodies.

11. The method of claim 10, wherein the antibody specifically binding active matriptase is selected from M69 and M123.

12. The method of claim 8, wherein the antibody which specifically binds to active matriptase is pecific for the conformational changes associated with the proteolytic activation of matriptase.

13. The method of claim 11, wherein at least one antibody which does not specifically bind to active matriptase is M32.

14. The method of claim 1, wherein the method further comprises detecting the presence and/or measuring the concentration in the sample of matriptase cognate inhibitor HAI-1 (M58).

15. The method of claim 1, wherein the biological sample is obtained by biopsy, nipple aspirate, or removal of body fluid that has come into contact with a malignant cell, cells of a pre-malignant lesion, or cells associated with a pathologic condition.

16. A method of treating malignancies, pre-malignant conditions, and pathologic conditions in a subject which are characterized by the activated form of matriptase comprising administering a therapeutically effective amount of an agent capable of blocking the activity of active matriptase.

17. The method of claim 16, wherein the malignancy and pre-malignant condition is a condition of the breast.

18. The method of claim 16, wherein the condition involves tissue remodeling; inflammatory responses, smooth muscle cell proliferation; cancer invasion or metastasis.

19. The method of claim 16, wherein the pre-malignant lesion is selected from the group consisting of: atypical ductal hyperplasia of the breast, actinic keratosis (AK), leukoplakia, Barrett's epiethlium (columnar metaplasia) of the esophagus, ulcerative colitis, adenomatous colorectal polyps, erythroplasia of Queyrat, Bowen's disease, bowenoid papulosis, vulvar intraepithelial neoplasia (VIN), and displastic changes to the cervix.

20. The method of claim 16, wherein the matriptase inhibiting agent is an antibody.

21. The method of claim 20, wherein the antibody is selected from M69 and M123.

22. The method of claim 16, wherein the malignancy, pre-malignant condition, or other pathologic condition, is in epithelial tissue or in a matriptase expressing tissue.

23. The method of claim 16, wherein the agent is capable of blocking the activation of matriptase by blocking the activity of an agent capable of inducing the activation of matriptase.

24. The method of claim 23, weherein the agent capable of inducing the activation of matriptase is compound comprising a lipid moiety.

25. The method of claim 23, wherein the agent capable of inducing activation of matriptase comprises a lipoprotein.

26. The method of claim 23, wherein the agent capable of inducing activation of matriptase comprises lysophosphartidic acid (LPA) or shingosine 1-phosphate (SIP).

27. A method of treating pathologic conditions in a subject which are characterized by the lack of the activated form of matriptase comprising administering a therapeutically effective amount of an agent capable of inducing activation of matriptase.

28. The method of claim 27, wherein the treatment involves wound healing.

29. The method of claim 27, wherein the agent comprises serum or an extract thereof.

30. The method of claim 29, wherein the serum comprises bovine; human; horse; goat; mouse; rat; rabbit; duck; or chicken serum; or a mixture thereof.

31. The method of claim 27, weherein the agent capable of inducing the activation of matriptase is compound comprising a lipid moiety.

32. The method of claim 27, wherein the agent capable of inducing activation of matriptase comprises a lipoprotein.

33. The method of claim 27, wherein the agent capable of inducing activation of matriptase comprises lysophosphartidic acid (LPA) or shingosine 1-phosphate (SIP).
